(19) United States
(12) Patent Application Publication
(10) Pub. No.: US 2011/0238177 A1
(43) Pub. Date: Sep. 29, 2011

(54) Biomechatronic Device

(76) Inventor: Joseph Anthony Farco, Hoboken, NJ (US)

(21) Appl. No.: 12/732,181

(22) Filed: Mar. 25, 2010

(51) Int. Cl. A61F 2/02

(52) U.S. Cl. 623/11.11

(57) ABSTRACT

A looped structure is rotatably coupled within the body of a prosthesis and rotates in response to fluid flowing across its looping to operate within the organism's internal functions.

### FIELD OF THE INVENTION

Disclosed are embodiments of the invention that relate to, among other things, obtaining medical advantages from natural fluid flow in a living organism.

### BACKGROUND

Fluids that flow through the body present a unique opportunity to extract additional energies separate and apart from those stored in the fluid itself.

The prior art has been unable to use natural fluid flows in an organism to produce mechanical systems and/or electrical systems that take full advantage of the organism's inner processes.

### SUMMARY OF THE INVENTION

A structure rotatably coupled to a prosthesis receives oncoming fluid flows. The structure rotates in response to contact with incoming fluid.

A looped wire rotatably coupled to a prosthesis receives oncoming fluid flows. The looped wire rotates in response to contact with incoming fluid.

A looped wire rotatably coupled to a prosthesis may be coated with a drug to disseminate the same during rotation of the same within an organism. The looped wire may also contain the drug to disseminate the same during rotation of the same within the organism.

A wire structure may rotate in a magnetic field found in or on the prosthesis and during such rotation receive an induced current from magnetic field. Current from the wire structure may be sent to at least one circuit element electrically coupled to the area of the prosthesis in which the wire is rotatably coupled.

### BRIEF DESCRIPTION OF THE DRAWINGS

FIGS. 1A-C illustrate a front view of a rotating structure facing fluid flow within an organism lumen according to exemplary embodiments of the present invention.

FIG. 2 illustrates another front view of a rotating structure facing fluid flow within an organism lumen according to other exemplary embodiments of the present invention.

FIG. 3 illustrates a pair of charge rings according to exemplary embodiments of the present invention.

FIG. 4 illustrates a commutator according to exemplary embodiments of the present invention.

FIG. 5 illustrates a commutator according to other exemplary embodiments of the present invention.

FIG. 6A illustrates a profile view of a charge ring pair according to exemplary embodiments of the present invention.

FIG. 6B illustrates a profile view of a charge ring pair according to other exemplary embodiments of the present invention.

FIGS. 7A-E illustrate operative profile views of a commutator according to several exemplary embodiments of the present invention.

FIG. 8 illustrates a commutator and cavity according to exemplary embodiments of the present invention.

FIG. 9 illustrates a commutator and cavity according to other exemplary embodiments of the present invention.

FIG. 10 illustrates a side view of a biomechatronic device facing fluid flow within an organism lumen according to exemplary embodiments of the present invention.

FIG. 11 illustrates a side view of an alternative biomechatronic device facing fluid flow within an organism lumen according to exemplary embodiments of the present invention.

FIGS. 12A-B illustrate a rotor conductive structure according to exemplary embodiments of the present invention.

FIGS. 13A-L illustrate conductive structure forms according to exemplary embodiments of the present invention.

FIGS. 14A-B illustrate conductive structure forms according to other exemplary embodiments of the present invention.

FIGS. 15A-B illustrate alternative conductive structure form and circuit interactivity according to exemplary embodiments of the present invention.

In the drawings like characters of reference indicate corresponding parts in the different figures.

### DETAILED DESCRIPTION

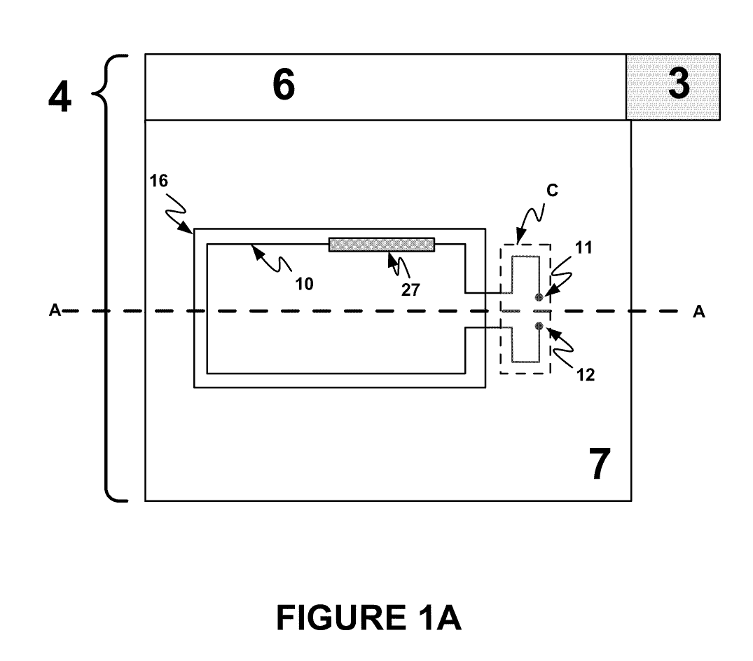

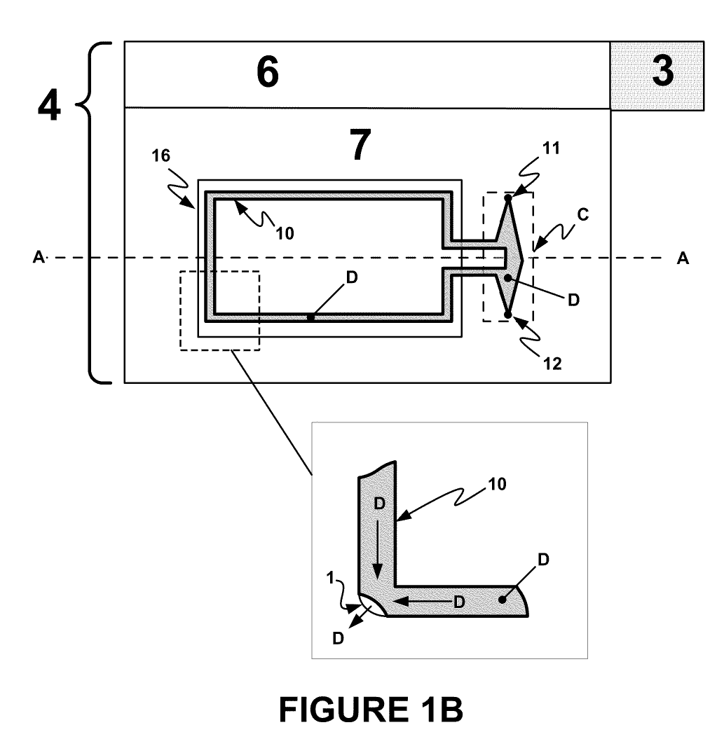

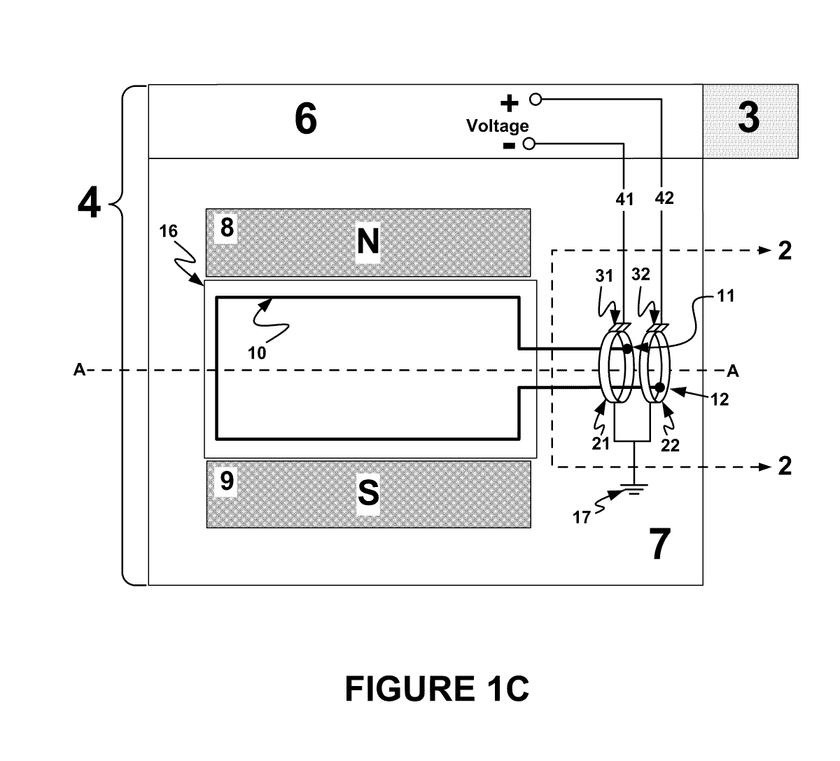

In the exemplary embodiment of the present invention illustrated in FIGS. 1A-C, a prosthetic 4 may be coupled to tissue 3 of an organism at tissue interface 6. According to this embodiment, prosthetic 4 comprises a platform 7 having one of the most proximal contact to the fluid flowing within tissue 3. Prosthetic 4 may be a graft, patch, sheet, suture, stent graft or other biologically or medically suitable artificial device that couples to tissue 3 of an organism. Accordingly, platform 7 may be any component of like or similar material to prosthetic 4 and may be attached to the same by sewing, screwing, molded formation, adhesion, welding or other coupling techniques known to those in the prosthetic device arts. Tissue interface 6 may be any site on prosthetic 4 that couples to tissue 3. The coupling of tissue interface 6 and tissue 3 may be made in any medically suitable fashion known to those skilled in the art. An exemplary tissue 3 according to the aforementioned embodiments of the present invention may be arteries, veins, organs or other fluid carrying lumen.

In a preferred embodiment, prosthetic 4 is a graft or stent graft, which is inserted into human vasculature either through a catheter or by surgical implantation. Alternatively prosthetic 4 is an artificial organ surgically attached within an organism. Platform 7 is an internal portion of the same luminar prosthetic extending distally towards the center of the lumen enclosing tissue fluid and is in direct contact with the fluid flowing there through. Tissue interface 6 is the surface of the prosthetic 4 that is in direct contact with the organism's biological vasculature.

In a prosthetic 4 that is a stent graft, platform 7 may possess a skeleton made of the same stent that makes up stent-graft 4. Alternatively, platform 7 may be made of a separate stent and graft that is sewn to the inside of stent-graft 4, adhered to stent-graft 4 or otherwise attached in any medically suitable fashion thereto. Alternatively, wire stent of stent-graft 4 may possess a wire structure that hangs down (e.g., a rectangular, circular or oval winding) extending distally toward the center of the stent structure while the remainder of the stent wire has a circular shape. This particular structure may be analogized to a tunnel with a portion of the tunnel wall extending towards the center of the tunnel from the ceiling of the tunnel (the stent wire or stent graft being the tunnel and platform 7 the tunnel extension). In that analogy, traffic through the tunnel is the equivalent to fluid flow through surrounding tissue 3.

According to the preferred embodiment in the form of a stent-graft, a descending wire making platform 7 may fold up when the stent is compacted into a catheter and open to its descending position when the stent-graft 4 is deployed. In the instance where the platform 7 wire is shape-memory metal, the deployed platform 7 will return to its descended position when the entire prosthetic 4 is deployed. This descended portion of the stent in stent-graft 4 may be coated with the same material as is stent-graft 4 or it may be covered with other biocompatible material depending on the intended use of the stent-graft 4 (e.g., denser polymers to keep the descended stent wire portion from migrating in response to fluid flowing through the tissue).

A wire-like structure 10 may be utilized to effect operation in the many embodiments of the present invention. Wire 10 may be made of any biologically suitable material (e.g., polymers, metals, ceramics, etc.) and may be solid or hollow and shaped in any way that permits operation for a given embodiment.

According to an exemplary embodiment of the present invention illustrated in FIG. 1A, wire 10 is rotatably coupled to platform 7. Wire 10 may also be a structure that is flat, fan shaped or any other shape that would move in relation to fluid flow across the surface. Wire 10 may be a looped wire whose loopings rotatably couple it to a cavity C within platform 7. Looped wire 10 has loopings that may permit it to rotate about axis A in an aperture 16 in platform 7. Looped wire 10 may be hollow or solid in shape. An exemplary looped wire 10 may be made of shape-memory metal. Alternatively, looped wire 10 may be a shape-memory polymer. In all cases, a covering 27 may cover looped wire 10. Covering 27 may be comprised of any biologically suitable material (e.g., biologically suitable polymers, drug, or combinations of materials). Covering 27 may be referred to in greater detail in conjunction with the discussions of further embodiments of the present invention herein.

The portion of wire-like structure 10 rotatably coupling it to platform 7 is sufficiently enclosed within a cavity C in platform 7 so that the structure 10 cannot fall out of platform 7. Structure 10 rotates substantially on an axis substantially perpendicular to tissue 3 fluid flow. Aperture 16 is shaped to permit rotation of structure 10 therein in response to fluid flow across the surface(s) of the structure 10 sections. Aperture 16 may preferably be a cut straight through platform 7 but may alternatively be only a partial cut through platform 7 surface (e.g., in embodiments where fluid flow through platform 7 may not be necessary). Where structure 10 is a looped wire, looped wire 10 comprises windings at wire ends 11 and 12 that may allow wire 10 to remain rotatably attached to platform 7 during operation. Wire ends 11 and 12 may be of a diameter and shape independent of wire 10. In this embodiment, looped wire 10 may also be made to rotate within an adequately sized aperture 16 in platform 7.

In a preferred embodiment, a “T” shaped loop formed by wire ends 11 and 12 of looped wire 10 is placed within a partial cavity of a portion of prosthetic 4 (e.g., an etched mold of polymer) and a next portion of prosthetic 4 having the remainder of the cavity etched therein being molded, glued or otherwise attached to the exposed partial cavity with the “T” wire looping found within. Together, the full cavity C is made to ensure no displacement of the looping during rotation of looped wire 10 in response to flow of fluid in tissue 3. Alternatively, wire ends 11 and 12 may be twisted in a cylindrical or helical configuration that may be similarly held within a cavity C appropriately shaped within platform 7.

In a preferred embodiment, a drug 27 that dissolves when in contact with tissue fluid 3 may cover looped wire 10 rotating within an aperture 16. According to this preferred embodiment, the rotation of drug-covered looped wire 10 acts as a type of time-release drug delivery system that permits drug dissolution over a period of wire rotation. According to this preferred embodiment, aperture 16 may only be cut partially into the surface of platform 7 and thereby merely catch fluid flowing through looped wire 10 coated with drug 27. Accordingly, build-up of fluid within tissue 3 behind looped wire 10 collected within aperture 16 of platform 7 may induce further dissolution and mixing of drug 27.

In the illustrative embodiment of the present invention in FIG. 1B, structure 10 may be hollow and contain a drug D therein. Structure 10 may also have at least one opening 1 from which drug D may exit into contact with the surrounding fluid environment. Preferably, openings 1 may be arranged at the corners of the structure 10 to allow exit of drug D from within. Drug D may exit structure 10 due to centrifugal forces exerted against the drug during rotation of structure 10 in tissue 3. In one embodiment, structure 10 may be a looped hollow tube made of any biologically suitable material. As the rotation of hollow looped tube 10 continues, vortex forces may also draw out drug D stored within the enclosed tube ends 11 and 12 which may be configured to act as a reservoir for drug D while also rotatably coupling tube structure 10 to platform 7 within cavity C. Alternatively, if structure 10 is made of a porous material, rotation of structure 10 may allow drug D to permeate through structure 10 to enter the fluid of the organism. In another embodiment, structure 10 may be a hollow shape in which a drug D is disposed.

In another preferred embodiment, looped wire 10 may contain an inner and outer diameter substantially similar to that of a syringe needle. Rotation of hollow looped wire 10 moves drug D disposed within wire 10 to exit from perforations 1 in the looped wire 10's outer diameter to interact with the tissue fluid in tissue 3. In this preferred embodiment, the rotation of a hollow wire 10 containing drug would also act as a time-release drug delivery system that allows drug to enter tissue 3 fluid as a result of centrifugal forces acting on the drug.

In another preferred embodiment, looped wire 10 may contain an inner and outer diameter and be made of a shape-memory polymer. Rotation of hollow polymer looped wire 10 moves drug D disposed within wire 10 to exit from perforations 1 in the looped wire 10's outer diameter to interact with the tissue fluid in tissue 3. In this preferred embodiment, the rotation of a hollow polymer wire 10 containing drug would also act as a time-release drug delivery system that allows drug to enter tissue 3 fluid as a result of centrifugal forces acting on the drug.

Referring to the exemplary embodiment of the present invention illustrated in FIG. 1C, platform 7 may comprise two oppositely polarized magnets 8 and 9 (shown in FIG. 1C as North magnet 8 and South magnet 9). Magnets 8 and 9 are separated and may be substantially parallel to one another. In one embodiment of the present invention, north magnet 8 is separated from south magnet 9 by aperture 16 in the platform 7. In operation, the previous embodiment may permit fluid flowing through tissue 3 to flow through aperture 16 which in turn allows said fluid to flow from one surface of platform 7 to the other (e.g., completely through platform 7). Preferably, fluid within tissue 3 flows through platform 7 in between magnets 8 and 9.

Magnets 8 and 9 may be made of any magnetic material known to those skilled in the art (e.g., AlNiCo, NiZn, FeCrCo, SmCo, NdFeB, Ferrite, etc.) and be dimensioned to reside within or attached to platform 7 as micro-magnets, thin magnetic films, foils or sheets having high magnetic-flux densities, such as, for example, those disclosed in U.S. Pat. Nos. 6,140,902; 5,858,126; 5,415703; 5,091,266; 4,921,763; 4,755,899, 7,335,229, the disclosures of which are completely incorporated herein by reference in their entirety. Those skilled in the art would understand that there are numerous other types of thin, compact, high magnetic-flux density material that may serve as magnets 8 and 9 or may otherwise create a desired magnetic field over a distance (e.g., spanning across aperture 16). It is envisioned for some embodiments, shape-memory magnetic alloys may also be used. Magnets 8 and 9 may be exposed to oncoming tissue fluid or embedded completely within platform 7.

According to FIG. 1C, a looped wire 10 that is electrically conductive may rotate about axis A between magnets 8 and 9 due to impacting fluid flows within a lumen formed of tissue 3. Wire 10 may be substantially parallel to magnets 8 and 9 and may rotate within aperture 16. Aperture 16 may be shaped to permit wire 10 to freely rotate between magnets 8 and 9 while also being either partially or completely within aperture 16. Wire 10 may also rotate freely in a given fluid stream without any portion of platform 7 surrounding its rotating wire surface. Wire 10 may be shaped such that it revolves in substantially one direction about axis A between magnets 8 and 9. According to this exemplary embodiment of the present invention, rotation of wire 10 between magnets 8 and 9 induces electrical current in wire 10.

In a preferred embodiment, platform 7 comprises a tab extending into fluid flow from a ceiling formed of a prosthetic 4 (e.g., a portion of an artificial heart, patch, stent, stent-graft, etc.). The prosthetic 4 and platform 7 are made of a biocompatible polymer. A section of platform 7 is removed leaving a through-hole 16 allowing flow of fluid within tissue 3 to which prosthetic 4 is attached. The remainder of the polymer material making up platform 7 will hold magnet 8 on one side of through-hole 16 and hold magnet 9 on the other side of through-hole 16. Magnets 8 and 9 may be partially exposed or completely embedded in the polymer material but are close enough to the edges of through-hole 16 to create a substantial magnetic field to traverse the through-hole space. Looped wire 10 rotates in through-hole 16 in platform 7 between magnets 8 and 9, the through-hole 16 being dimensioned substantially to fit around the dimensions of the outermost looping of looped wire 10. Looped wire 10 rotates by having one of its loops rotatably coupled within a cavity in the polymer material in platform 7. Each time a looped surface of wire 10 crosses a magnetic field, a current is induced in the section of wire 10.

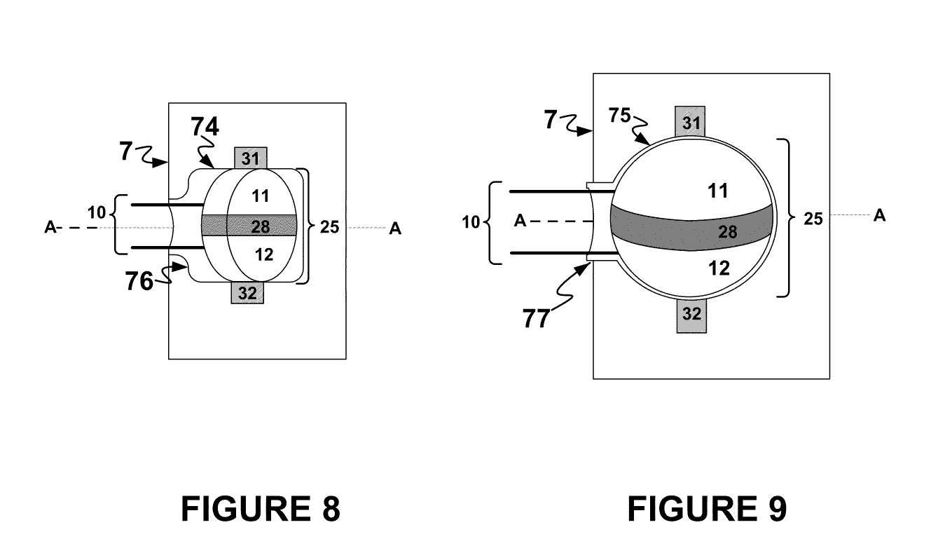

In another preferred embodiment, platform 7 comprises a dangling undulation of stent wire extending distally toward the center of the stent, the undulating stent wire forming the structure is covered by graft material. A portion of the graft material covering the undulation is cut to make slit 16 from platform 7 with sufficient amount of graft material to hold magnets 8 and 9 on either side of slit 16. Looped wire 10 will rotate in slit 16 of platform 7 and its loopings will be rotatably coupled within the surrounding graft material such that looped wire 10 will not exit platform 7 in response to oncoming fluid flow through tissue 3. In this preferred embodiment, additional layers of graft material may be sewed around wire 10's loopings to form a rotatable couple cavity C enclosing the loopings and rotatably coupling wire 10 to platform 7. A preferred rotatable couple cavity C may comprise heat molded graft material or a cavity C structured out of surrounding or independent stent metal. Looped wire 10 rotates on an axis substantially parallel to magnets 8 and 9 as fluid flows within tissue 3 and prosthetic 4 and through platform 7 at slit 16. For a rotatable couple cavity made of stent metal of the type used in a stent-graft 4, it is preferred that the metal be suitable for use in the desired medical procedure (e.g., self-expansion when deployed in an artery or vein). The stent metal may be wound so to engulf looped wire 10 and permit it to rotate, e.g., a cylindrical cage of stent metal from stent 4 shaped in substantially similar form to item 7 in FIG. 8 or 9.

According to the exemplary embodiment of the invention illustrated in FIG. 1C, wire ends 11 and 12 rotatably couple a looped wire 10 to platform 7. According to this exemplary embodiment, wire 10 may be made of any electrically conductive, biocompatible material. In one embodiment of the present invention, wire 10 may be made of Nitinol or other electrically conductive shape-memory metal. In another embodiment, wire 10 may be made of a corrosion resistant conductive metal, such as for example, the type of metal used in intraluminal operations of the type disclosed in U.S. Pat. No. 5,170,802 and U.S. Patent Application Publication No. 2005/0080346 (U.S. patent application Ser. No. 10/939,684) published Apr. 14, 2005, the disclosures of which are hereby completely incorporated by reference in their entirety. Wire 10 may be made of any electrically conductive material while also being substantially covered by any form of biocompatible material known to those skilled in the art. According to this embodiment, covering 27 may substantially cover electrically conductive portions of wire 10 during operation of the disclosed embodiments of the present invention. However, covering 27 may leave exposed wire ends 11 and 12 of wire 10 to permit their rotatable coupling within the cavity of platform 7.

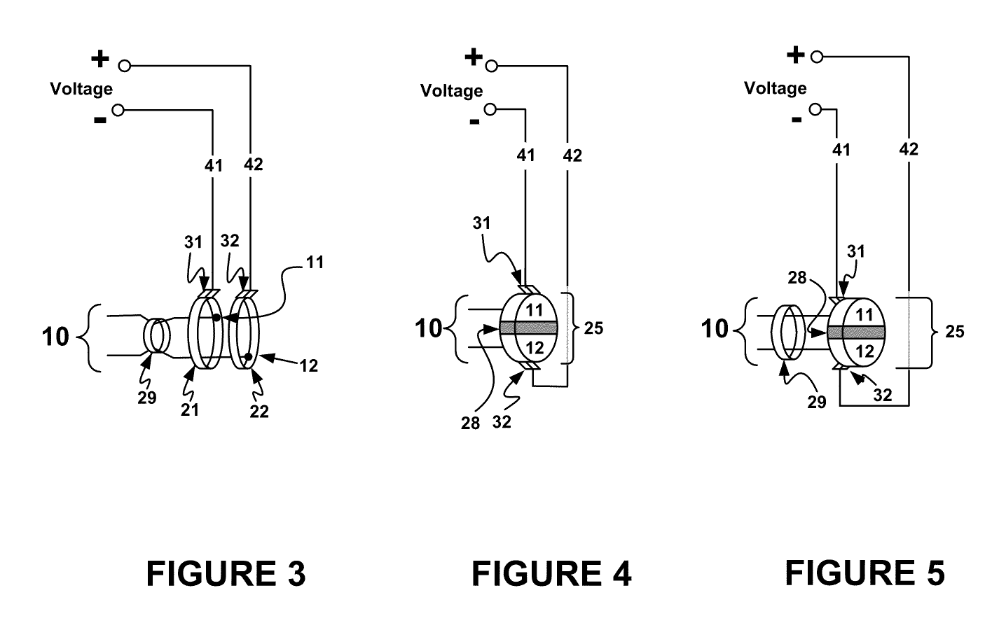

When configured to conduct charge created in looped wire 10, the embodiment of FIG. 1C illustrates wire ends 11 and 12 rotatably coupling looped wire 10 to a charge section 2 to collect the created charge. In this way, charge section 2 replaces cavity C of FIGS. 1A and 1B. The coupling of wire ends 11 and 12 acts as an electrical connection between wire 10 and charge section 2. Thus, in this embodiment, wire ends 11 and 12 simultaneously rotatably couple and electrically connect wire 10 to charge section 2 in platform 7. Rotation of wire 10 in the aperture 16 between magnets 8 and 9 induces current in wire 10 that may be sent into charge section 2 and platform 7 through wire ends 11 and 12, which also act as the rotatable coupling for wire 10 to induce such current. Charge section 2 may embody numerous forms of charge reception within any portion of prosthetic 4 and may comprise, for example, the components and designs depicted in FIGS. 3-9. A circuit (not shown) connected to charge section 2 may receive charge from the induced current caused by rotation of wire 10 between magnets 8 and 9 due to flow of fluid in tissue 3 over wire 10 (e.g., blood, water, air, etc.)

An exemplary equation illustrative of wire 10's operation according to the various embodiments of the present invention may be as follows:

ξ(t)=NBab2πf sin(2πft) Equation 1
ξ(t) represents the voltage at any given instant in wire loop
N represents the number of turns of wire
B represents the magnetic field in which the turns of wire rotate
a represents the length of each wire turn
b represents the width of each wire turn
f represents the frequency of rotation of turns of wire

Variability in any one of the structural parameters of the looped wire, the magnetic field strength or frequency of rotation may be desirable based on the functionalities required of the embodiments of the present invention. In one embodiment, altering the characteristics of looped wire 10 best achieves optimized values of voltage according to Equation 1 because fabrication and manufacturing techniques (to be described) may produce advantageous values for N, a and b in Equation 1 without significant cost or space losses in prosthetic 4. Placement of the embodiments of the present invention at active parts of the organism may have direct effects on f in Equation 1. Therefore, in a preferred embodiment, it is advantageous to use known micro-wire fabrication techniques to create numerous wire loops in a small area of wire while also placing those loops in high-traffic fluid locations within the organism.

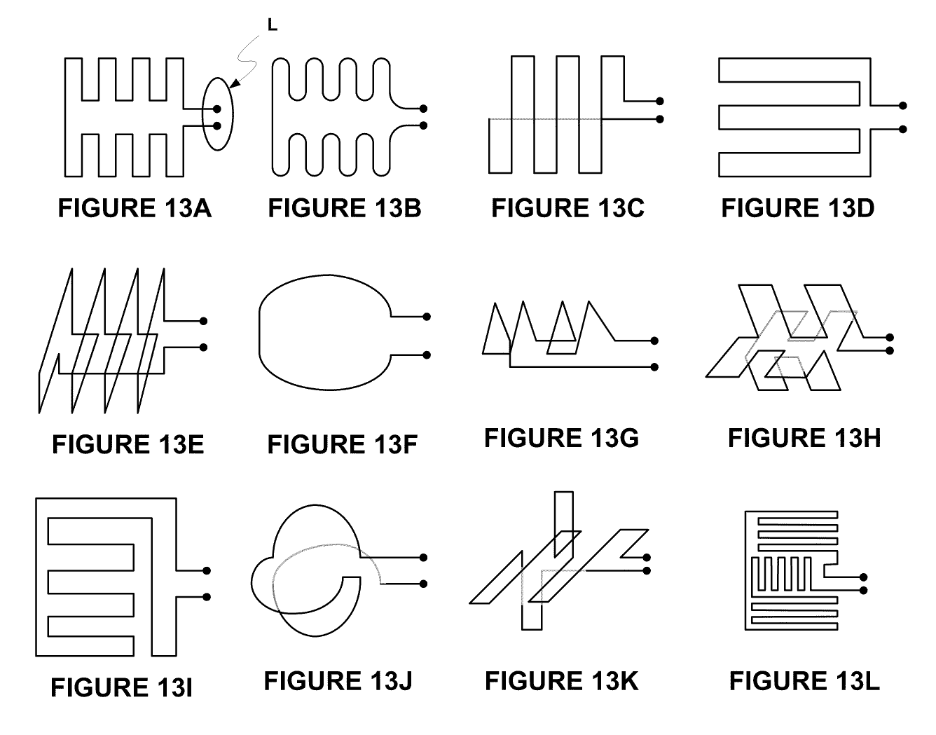

According to the embodiments of the present invention according to FIG. 1A, 1B or 1C, wire 10 may be shaped, bent and/or looped in any number of possible ways (see, for example, FIGS. 13A-L, 14 A-B and 15A-B and the embodiment disclosures related thereto) that would permit the operation of the other embodiments of the present invention herein disclosed. Wire 10 may be shaped, bent and/or looped to account for the intraluminal insertion of prosthetic 4 into or adjacent to tissue 3 (e.g., preparation of stent-graft 4 for expansion in a lumen, screwing prosthetic 4 to a bone or cartilage surface, sewing graft 4 to tissue 3, etc.) Looped wire 10 may be shaped, bent and/or looped according to mechanical bending processes, micro-depositing conductive metals into molds, molding metals or polymers into a preferred looping in a pre-fabricated mold, extruding the metal or polymer in a heated state to cool into a final looped state, laser cutting a metal substrate, etching, stamping, soldering wire to an acceptable board surface and either removing the surface or keeping the wire and surface for use in the body, and any other techniques known to those skilled in the art. Laser cutting is a preferred fabrication method as it can produce favorable wire structures according to Equation 1.

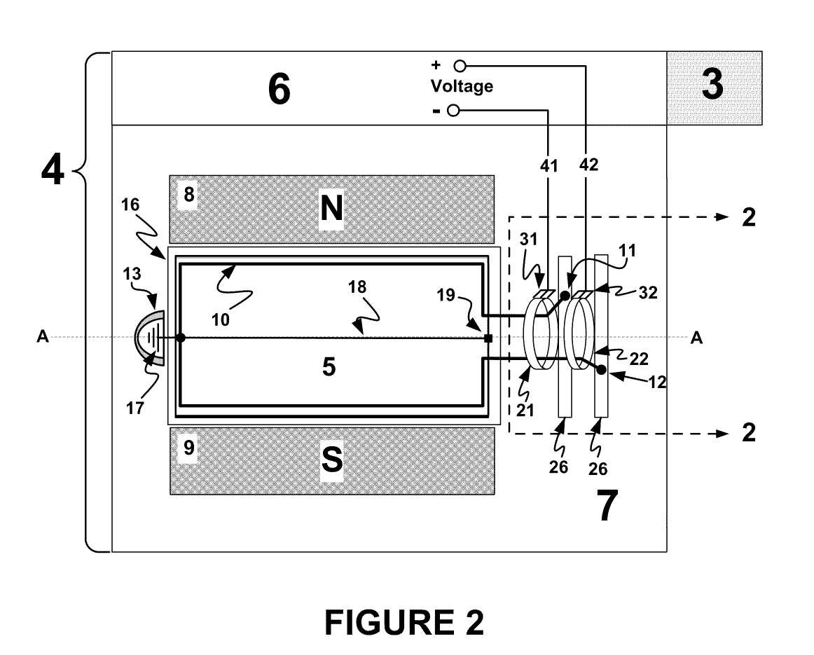

If the formation of a given configuration or looping of looped wire 10 requires additional support, covering 27 may be utilized to add to the integrity of the looped wire 10 configuration (e.g., resist easy bending in face of oncoming tissue 3 fluid). In a later embodiment, it will be shown that looped wire 10 may be placed within a solid construct that will ensure the integrity of the looped wire configuration (e.g., item 5 of FIG. 2). Thus, covering 27 may be both dissolvable (in the case of a drug) and structure fortifying. However, in the case of permanent covers 27, the rotating structure 10 may take on a shape, which is a combination of both the interior wire shape and the cover 27 shape.

In a preferred embodiment, a looped wire is coupled to an expanded polytetrafluoroethylene (ePTFE) graft prosthetic having a 10.0 mm diameter placed within a blood vessel. A 9.0 mm diameter Nitinol wire is encapsulated in the ePTFE graft prosthetic and forms a “hanging sign” wire arrangement covered with a PVC film (the platform). The PVC platform extending from the wall of the prosthetic is approximately 3.6 mm in height, 4.0 mm in width and 3.0 mm in depth. Within the PVC platform resides two AlNiCo micro-magnets with dimensions of 2.8 mm width, 1.1 mm height and 1.0 mm depth on either side of a 2.0 mm by 0.875 mm rectangular slit through the PVC platform extension. A looped wire 1.0 mm wide by 0.75 mm high consists of a discrete time-signal shaped loop configuration including 8 turns of wire where each turn is 0.2 mm wide and 0.3 mm long. Looped wire 10 is preferably laser cut from a 0.125 mm thick Nitinol sheet to create a looped formation equivalent to a mechanically bent 36-gauge wire. The wire may be covered with a lightweight polymer film spray of Polydiaccrylate-BaTiO₃ to maintain integrity of the small gauge wire in the face of flood flows. Wire ends 11 and 12 would not be spray coated with the aforementioned polymer as they include an additional 0.75 mm extension into the PVC platform to connect the looped wire 10 to a cavity containing charge components in the PVC platform.

With reference to the illustrative embodiment of FIG. 1C, charge section 2 comprises slip rings 21 and 22 and electrical ground 17. Contacting each slip ring 21 and 22 are brush contacts 31 and 32 respectively. From each of brush contacts 31 and 32 stems circuit wiring 41 and 42, respectively, connecting slip rings 21 and 22 to a circuit (not shown). According to this embodiment of the present invention, slip rings 21 and 22, brush contacts 31 and 32 and circuit wiring 41 and 42 are electrically conductive and biocompatible materials (e.g., wires and conductive foils). Brush contacts 31 and 32 may be made of brass, but may be made of other biologically suitable materials. Alternatively, brush contacts 31 and 32 may be made of other materials that have enhanced longevity in the fluid flowing within tissue 3. Electrical ground 17 may ground current flowing in charge section 2 in any portion of platform 7 or even in other parts of prosthetic 4. For other embodiments, charge section 2 may comprise components that are made of conductive, shape memory metal (e.g., Nitinol).

Each of elements of charge section 2 may be integrated with platform 7 in a manner that would not disturb their normal operation as disclosed herein. According to certain embodiments, elements of charge section 2 may be embedded within platform 7. According to other embodiments, charge section 2 elements may be affixed to platform 7. In yet other embodiments, charge section 2 may possess elements that are moveably lodged within spaces within platform 7.

In a preferred operation according to an exemplary embodiment of the present invention illustrated in FIG. 1C, fluid in tissue 3 contacting wire 10 causes wire 10 to rotate between magnets 8 and 9 inducing current in wire 10. Simultaneously, wire 10 is rotatably coupled to platform 7 by its wire ends 11 and 12 which double as electrical contacts to charge section 2. The current induced in rotating wire 10 flows from wire end 11 to wire end 12 creating a potential between both wire ends. Simultaneously, wire ends 11 and 12 are both rotatably and electrically coupled to slip rings 21 and 22, respectively, within prosthetic 4. As induced current is generated by the rotating wire 10, the rotating wire ends 11 and 12 transfer wire 10 induced current flow to slip rings 21 and 22, respectively. The current in slip rings 21 and 22 is translated to brush contacts 31 and 32, respectively, which in turn translate that current to circuit wire 41 and 42, respectively. Any and all charges in the slip rings, contacts or circuit wires have electrical attachment to ground 17. According to this exemplary embodiment, wire 10 and its ends 11 and 12 may be electrically coupled to a circuit (not shown) through any one of the components in charge section 2.

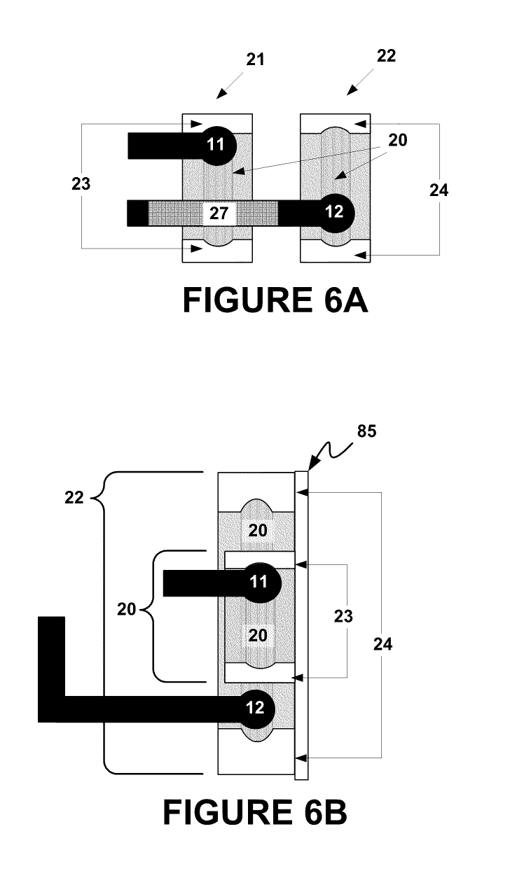

Each of the components of charge section 2 in FIG. 1C may be fixed or moveable vis-à-vis wire ends 11 and 12. In one embodiment, wire ends 11 and 12 may be rotatably coupled within slip rings 21 and 22 respectively (e.g., wire end 11 rotates in slip ring 21 and wire end 12 rotates in slip ring 22). Slip rings 21 and 22 may be shaped in any manner to permit rotatable coupling of wire ends 11 and 12 respectively thereto, e.g., in the shape of a circle or ellipse with a width to receive surface contact between inner ring surface and wire end 11/12. A more detailed view of slip rings 21 and 22 may be had with reference to FIGS. 6A-B. In an alternative embodiment, wire ends 11 and 12 may be integrated with and joined to slip rings 21 and 22, respectively, such that the combined slip ring and wire ends 21/11 and 22/12 may each rotate as one construct (or pair of constructs) due to rotation of wire 10. According to this embodiment, the combined slip ring and wire end combinations 11/21 and 12/22 rotate in contact with fixed brush contacts 31 and 32 respectively. It should be appreciated that this embodiment provides slip ring/wire end structures that resist displacement of wire 10 outside of platform 7.

As previously disclosed with respect to other embodiments, platform 7 may be formed to permit operative rotation of each wire/slip ring combination and also preclude such wire/slip ring combination from being able to exit platform 7 while in use (e.g., a bottle neck cavity in platform 7 providing a smaller opening in platform 7 for entering wire ends 11 and 12 which cannot also fit slip rings 21 and 22 and a larger opening for slip rings 21 and 22 to allow for rotation of wire/slip ring combinations 11/21 and 12/22).

Referring to FIG. 3, slip rings 21 and 22 may be preceded by at least one locking ring 29 around which wire ends 11 and 12 frictionally couple. The locking ring 29 may be integrated with platform 7 or may be adhesively joined thereto. Locking ring 29 may be made of any suitable biocompatible material. The various embodiments of the slip ring friction coupling described above are similarly applicable to locking ring 29. However, according to this embodiment of the present invention, because locking ring 29 need not be an electrical conductor, it may take the form of other friction coupling arrangements, e.g., PVC rings, rubber rings, etc.

Brush contacts 31 and 32 may be stationary within platform 7 or prosthetic 4 to collect the charge translated from wire ends 11 and 12. According to this embodiment, brush contacts 31 and 32 may be wires connecting slip rings 21 and 22 to a circuit (not shown). Brush contacts 31 and 32 may be substantially similar in shape and size to slip rings 21 and 22 (e.g., friction fittings or receptive contours for receiving slip rings 21 and 22 and/or contours for receiving wire ends 11 and 12). In a preferred embodiment, brush contacts 31 and 32 may be biologically compatible conductive material slidingly coupled to slip rings 21 and 22.

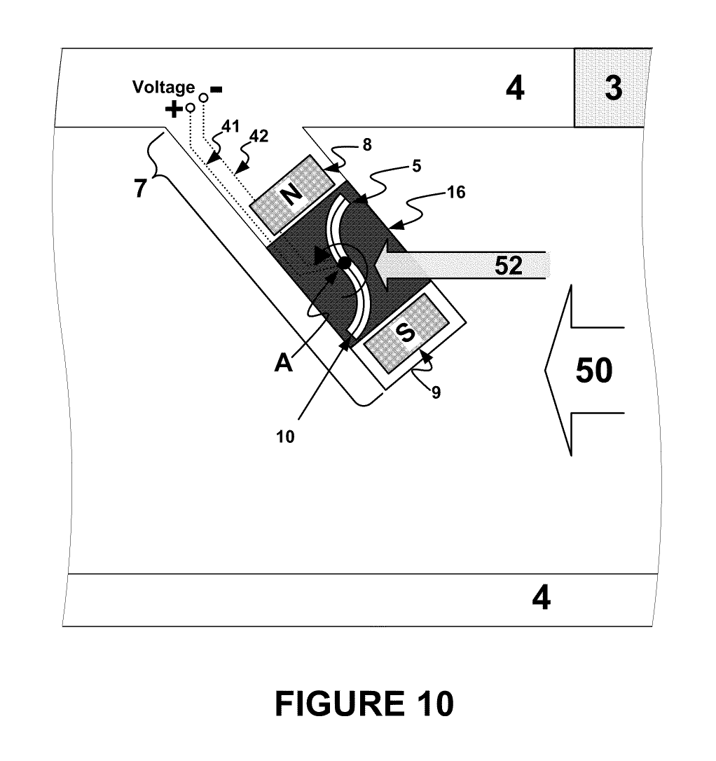

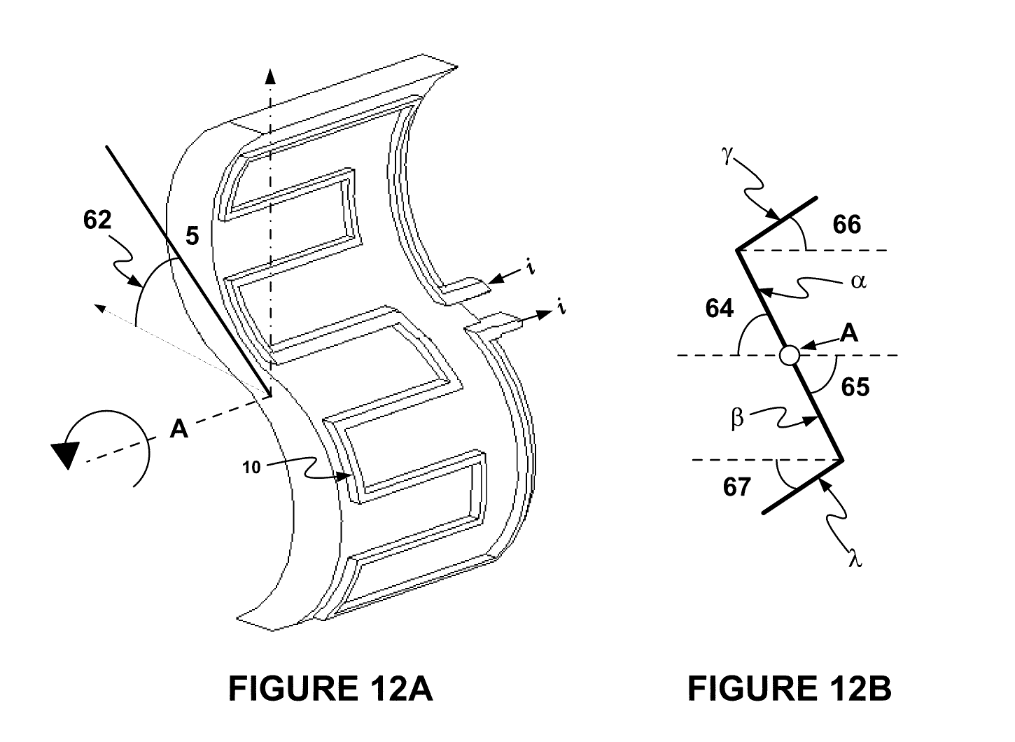

Referring now to the illustrative embodiment of the present invention in FIG. 2, a rotor 5 may comprise wire 10 (with or without covering 27) and be shaped either according to the shape of wire 10 or an alternative shape that induces rotation when fluid impacts the surface. Rotor 5 may cover wire 10 like covering 27 but may also cover gaps formed by the loops, twists and turns of wire 10 according to other embodiments of the present invention. An exemplary rotor 5 may be shaped as a flat paddle or have a substantially “S” shape. A more detailed discussion of the shape of rotor 5 will be made with respect to FIGS. 10, 12A-B and 14A-B. Rotor 5 may be made of any material that will not interrupt induction of current in wire 10 according to the other embodiments of the present invention. In one exemplary embodiment, rotor 5 may be made of a porous material (e.g., ePTFE), textured surface material or material with openings through the surface (e.g., cut or etched silicone, ePTFE or bio-plastic). Such materials may increase fluid friction and generate greater rotation of rotor 5 and consequently, wire 10. In another exemplary embodiment, rotor 5 may be made of a substantially smooth surface such, for example, a vinyl or plexi-glass. In yet another exemplary embodiment, rotor 5 may be made of fabrics, ceramics or electric ribbon (e.g., ribbon such as the type manufactured by Hirose Electric Co., Ltd.) Numerous other materials suitable for use as a rotor 5 are envisioned according to the various embodiments of the present invention.

Also depicted in the illustrative embodiment of the present invention in FIG. 2 is a wire support 18 and anchor 19. In conjunction with the embodiments of FIGS. 1A and B, wire support 18 and anchor 19 may be additional extensions of wire structure from looped wire 10 extending within the wire loop or into platform 7. According to other embodiments of the present invention, wire support 18 may be any portion of wire 10 not used to provide current to charge section 2 (e.g., a metallic wire of higher gauge value or flexibility, a non-metallic wire, etc.). The end of wire support 18 is anchor 19. Anchor 19 may adhere wire support 18 to a surface of rotor 5. Wire support 18 and anchor 19 may assist rotation of rotor 5 and wire 10 in turbulent fluids or prevent unwanted stress on rotor 5 or wire 10. Wire support 18 may be shaped in any fashion that provides additional support to rotor 5 and/or wire 10 to operate according to the other embodiments of the present invention.

In FIG. 2, wire 10 may be grounded by ground 17 in a location apart from charge section 2. In an exemplary embodiment of the present invention, wire 10 may be grounded by ground 17 coupled within pocket 13 formed in platform 7. According to this embodiment, ground 17 may serve as both an electrical ground for the induced current in wire 10 (as a result of processes herein described) and as a rotational joint for wire 10 in platform 7. In this exemplary embodiment of the present invention, wire 10 is rotatably coupled to platform 7 through ground 17 in pocket 13 and by wire ends 11 and 12 in charge section 2. Similarly, rotor 5 may be rotatably coupled to platform 7 through ground 17 in pocket 13 and by wire ends 11 and 12 in charge section 2.

In these embodiments, ground 17 may be shaped to rotate within pocket 13 but not exit pocket 13 (e.g., pocket 13 substantially encloses the largest surface of ground 17 and allows the lesser surface, the surface connecting wire 10 to platform 7 across aperture 16, to rotate upon rotation of wire 10, etc.). In a preferred embodiment, pocket 13 encloses substantially all of ground 17 except for an opening substantially the same size as the thickness of wire 10 to ensure rotation of wire 10. In another embodiment, pocket 13 may also lock ground 17 with a friction fit similar to that of locking ring 29 described in previous embodiments. Other pocket forms may be utilized to restrict ground 17 and/or wire 10 from exiting pocket 13 (e.g., bottleneck form allowing for entry of ground wire 17 into the smaller opening but precluding exit of ground 17 end located in larger opening, etc.). In yet another embodiment, ground 17 may rotatably couple looped wire 10 to pocket 13 according to the disclosed looped wire 10 couplings described above (e.g., “T” shaped coupling). Other shapes suitable to keep ground 17 from exiting pocket 13 can be envisioned without departing from the scope of the present invention.

Referring to an exemplary charge section 2 as illustrated in FIG. 2, slip rings 21 and 22 may be integrally placed in platform 7 and wire ends 11 and 12 establish the electrical connection with their respective slip rings by forming friction couplings within or about slip rings 21 and 22. According to this exemplary embodiment of a charge section 2, wire end 11 and/or 12 may be bent about the distal edges of slip rings 21 or 22, respectively, to prevent substantial displacement of wire 10, wire end 11 and/or wire end 12. According to this exemplary embodiment, slits 26 in platform 7 made adjacent to slip rings 21 and/or 22 may permit bent wire ends 11 and/or 12 to freely rotate while bent about distal edges of their respective slip rings. In another variation of this exemplary embodiment, slip rings 21 and/or 22 may have round toroidal shapes around which their respective wire ends 11 and 12 bend. The slits 26 may be shaped to allow operation of the disclosed embodiments of the present invention (e.g., shaped according to the length and bending of wire ends 11 and 12).

FIGS. 3, 4 and 5 each show exemplary embodiments of charge section 2 according to the present invention. FIG. 3 is an illustrative embodiment of a charge section 2 interacting with rotating wire 10. According to this embodiment, charge section 2 comprises a lock ring 29 frictionally coupling wire ends 11 and 12 to rotatably and electrically contact their respective slip rings 21 and 22. Brush contacts 31 and 32 are electrically contacting slip rings 21 and 22 to transfer current induced in wire 10 to circuit wires 41 and 42. The current induced in wire 10 creates a charge potential which may be used as a voltage or current source. According to one embodiment of the present invention according to FIG. 3, lock ring 29 may be embedded in platform 7 to prevent displacement of wire 10, wire ends 11 and 12 and/or slip rings 21 and 22 during rotation. Slip rings 21 and 22 may be separate and outside of platform 7 according to the embodiment of FIG. 3. A charge section 2 according to the illustrative embodiment of FIG. 3 may be used to generate alternating current (AC) for the circuit (not shown) to which it is electrically coupled via circuit wires 41 and 42.

In the exemplary embodiment of the present invention according to FIG. 4, wire ends 11 and 12 are coupled to insulator 28 to form commutator 25. Insulator 28 may be any known insulator (e.g., a ceramic or air space). Insulator 28 may be dimensioned according to the space of the charge section 2 components. Commutator 25 may be any shape capable of being rotatably coupled to brush contacts 31 and 32 (e.g., rotating within a contour of brush contacts 31 and 32, rotating on the surfaces of brush contacts 31 and 32, etc.) lodged within platform 7. According to this exemplary embodiment, commutator 25 will rotate as a single mass as wire 10 and its respective wire ends 11 and 12 rotate. As brush contacts 31/32 come in contact with insulator 28, the induced current from wire 10's rotation between magnets 8 and 9 will temporarily cease to transfer from wire 10 to brush contacts 31/32. In subsequent iterations of this event, this operation of commutator 25 according to this exemplary embodiment of the present invention may generate direct current (DC) for the circuit (not shown) connected to circuit wires 41 and 42.

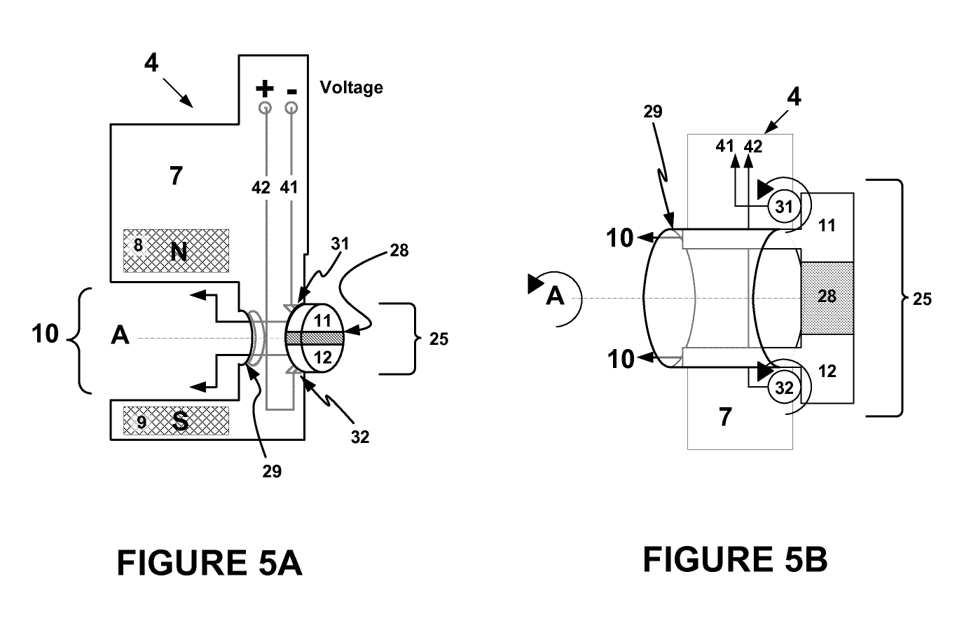

According to the embodiment of FIGS. 4 and 5, wire ends 11 and 12 need not form substantially disk-like constructs but may be bent in a crescent-like shape that will rotate within a substantially circular cavity in platform 7. As described above, the crescent wire ends 11 and 12 may be bent, formed, etched or laser cut according to known processes. For example, wire ends 11 and 12 may form oppositely facing “C” shapes separated by a gap to act as an insulator 28. Alternatively, the same oppositely facing “C” shapes wire ends 11 and 12 can be connected by a layer of polymer 28.

In the exemplary embodiment of the present invention according to FIG. 5, wire ends 11 and 12 are coupled to insulator 28 to form commutator 25. Additionally, wire ends may pass through a locking ring 29 that is smaller in diameter than commutator 25. In one variation of this embodiment of the present invention, locking ring 29 may frictionally couple wire ends 11 and 12 (e.g., as shown in FIG. 3). In another variation, locking ring 29 may preclude displacement of commutator 25 during rotation of wire 10. According to either variation, locking ring 29 may be integrated with platform 7. According to this embodiment, brush contacts 31 and 32 may similarly be integrated with platform 7 to allow exterior rotation of commutator 25. Alternatively, as previously disclosed, each of the elements of the exemplary embodiment of charge section 2 may be integrated with platform 7. In a preferred embodiment of the present invention according to FIG. 5, commutator 25 may rotate so that its larger face retains a rolling contact with brush contacts 31/32 located within platform 7 (e.g., brush contacts 31/32 are parallel to rotation axis A, etc.). In a preferred embodiment, brush contacts 31/32 are rotating ballpoint electrical connections (e.g., like the ball of a ball-point pen) that may rotate in contact to commutator 25 to ensure longevity of contact over long periods of use.

FIG. 6A illustrates an exemplary embodiment of the wire ends 11 and 12 rotatably and electrically coupling wire 10 to slip rings 21 and 22 respectively. According to this embodiment, the series location of slip rings 21 and 22 may cause one of wire ends 11 or 12 to have to go through one slip ring and contact another. In the illustrative embodiment of FIG. 6A, wire end 12 may pass through slip ring 21 (which is the respective rotatable, electrical couple for wire end 11) and rotatably and electrically couple to slip ring 21. In one embodiment of the present invention, covering 27 may substantially cover wire end 12 to avoid electrical contact between wire end 12 and slip ring 21.

As shown in the illustrative embodiment of the present invention in FIG. 6B, slip ring 21 may be concentric and coplanar with slip ring 22 where the diameter of one slip ring is greater than the other to permit simultaneous rotation of wire ends 11 and 12 without contact. According to the embodiment of the present invention depicted in FIG. 6B, concentric slip rings 21 and 22 are held in their coplanar, concentric locations by integration with platform 7 or by adhesion or integration with a non-conductive material wall 85. The embodiments of the present invention envision use of biocompatible adhesives such as, for example, 50 polytetrafluoroethylene, polyurethane, polyethylene, polypropylene, polyamides, polyimides, polyesters, polypropylenes, polyethylenes, polyfluoroethylenes, silicone fluorinated polyolefins, fluorinated ethylene/propylene copolymer, perfluoroalkoxy fluorocarbon, ethylene/55 tetrafluoroethylene copolymer, and polyvinylpyrolidone. In one embodiment, material wall 85 may be a cavity wall of platform 7. Alternatively, material wall 85 may be any other known wall formed in prosthetic 4.

Referring again to FIG. 6A, thickness 23 of slip ring 21 may be shaped to receive within it the shape of wire end 11. In one embodiment, thickness 23 may possess a groove 20 shaped to receive wire end 11 and frictionally couple wire end 11 to slip ring 21. Groove 20 may also provide wire end 11 rotational and electrical coupling with slip ring 21 as per other embodiments of the present invention. FIG. 6A also illustrates thickness 24 of slip ring 22 being shaped to receive within it wire end 12. Similar to the embodiment of wire end 11 and slip ring 21, slip ring 22 provides a groove 20 that frictionally couples wire end 12 within the thickness 24 of slip ring 22. According to the exemplary embodiment of charge section 2 according to FIG. 6A, groove 20 of slip ring 22 and groove 20 of slip ring 21 may differ in geometry to keep wire ends 12 and 11 respectively from displacing during rotation. Similarly, thickness 24 may be different from thickness 23 to provide different stress support during operation of wire 10 (e.g., each thickness may have varying friction coefficients, etc.). The frictional coupling of grooves 20 and wire ends 11 and 12 may be achieved by pre-bending the wire ends and forcing them into a restricted bend constrained within rings 21 and 22.

In a preferred embodiment, wire ends 11 and 12 each are bulbous (e.g., spherical) nubs of conductive biocompatible metal no more than 0.1 mm in diameter. Slip rings 21 and 22 are Nitinol rings of 0.5 mm thickness affixed in a tubular TFE platform 7. The rings are concentric with their centers on looped wire 10 rotation axis A but are spaced apart from one another by a small gap (approximately 0.25 mm in length). The internal surface of each ring has a carved channel 20 about the circumference of the inside surface. The proximal portions of the channels 20 face looped wire 10 (e.g., they face the position closest to exiting platform 7 and encountering aperture 16). The proximal portion of channel 20 in slip ring 21 has a higher surface protruding from the thickness 23 of slip ring 21 than does the distal portion of channel 20 in slip ring 21. In this way the wire end 11 fits within channel 20 of slip ring 21 but is precluded from proximal displacement by the “hill” formed in the thickness 23 therein. Wire end 12 may be fitted within channel 20 of slip ring 22 located even deeper within tubular platform 7. However, in this preferred embodiment, both the proximal and distal portions of channel 20 formed in thickness 24 are embracing wire end 12. In this embodiment, wire end 12 is the anchoring focus for rotating looped wire 12 because it is the wire end that goes deepest into platform 7. Wire end 11 could also act as the anchoring focus. The thickness 24 of slip ring 22 is suited to increase friction at the rotatable, electrical couple of wire end 12 and channel 20 of slip ring 22. This thereby reduces the possibility of wire end 20 coming loose during operation of the looped wire 10 in tissue flood flow.

The same preferred embodiment described above to a separately spaced concentric slip ring 21/22 arrangement may also be had for a concentric, coplanar slip ring 21/22 arrangement. However in such a preferred embodiment, having only “hills” formed at the proximal portions of channels 20 of slip rings 21/22 should be sufficient to prevent migration of either wire ends 11, 12 or looped wire 10 during operation.

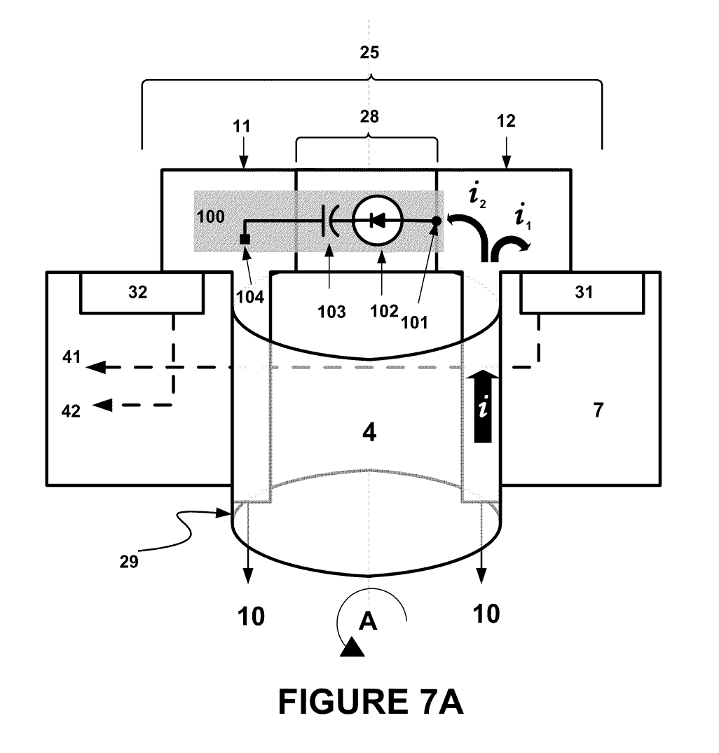

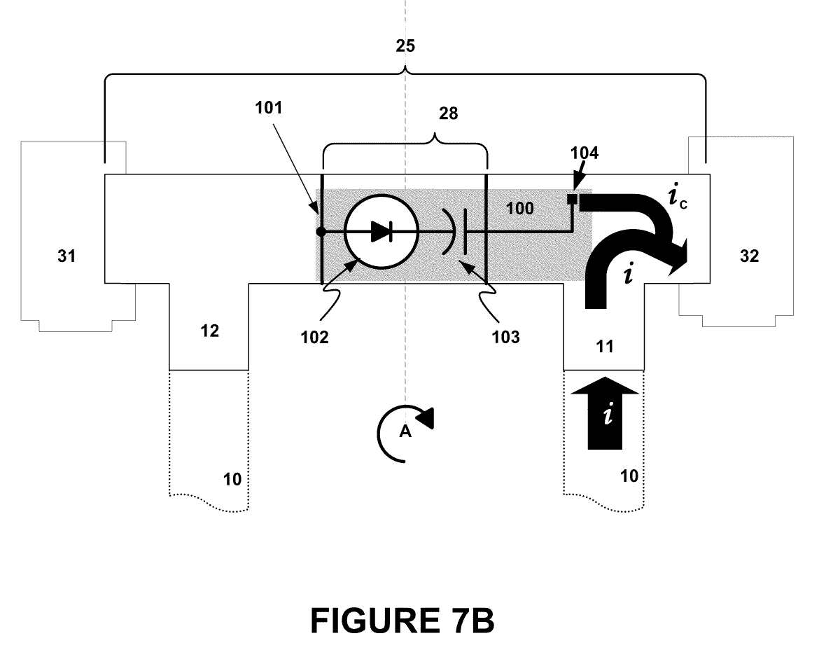

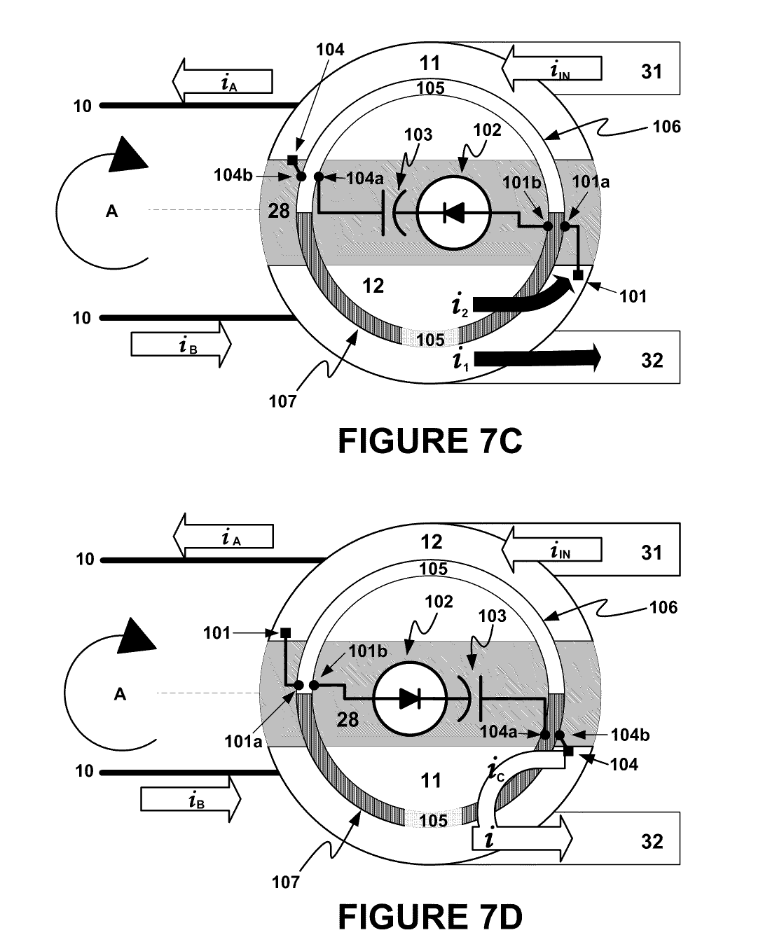

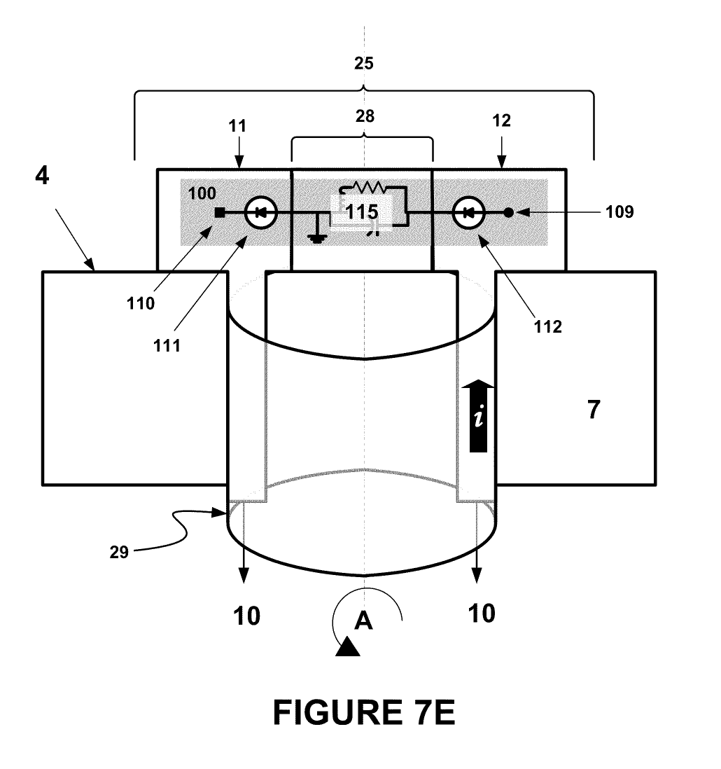

FIG. 7A depicts an exemplary commutator embodiment according to the present invention. An exemplary commutator 25 may be substantially cylindrical or spherical and may comprise a hemisphere occupied by wire end 11, a hemisphere occupied by wire end 12 and an equator comprised of insulator 28. On either face of wire end 11 and 12 are brush contacts 31 and 32 respectively. According to this exemplary embodiment, as commutator 25 rotates counter clockwise about axis A, brush contacts 31 and 32 will contact the surface of commutator 25 facing wire 10. In this embodiment, however, insulator 28 comprises at least one circuit 100 comprising any number and type of known electrical components (e.g., resistors, capacitors, transistors, transformers, amplifiers, inductors, diodes, switches, voltmeters, ammeters, etc.) known to those skilled in the art. Circuit 100 may be used to perform various operations (e.g., send signals, measure data, collect charge, etc.) according to embodiments of the present invention. In one embodiment, circuit 100 may be configured to collect charge during one time period and dissipate collected charge in a subsequent time period. The circuit 100 in insulator 28 may be substantially free of contact with brush contacts 31 or 32. According to this exemplary embodiment of the present invention, as commutator 25 rotates counterclockwise about axis A, the portions of insulator 28 comprising one circuit 100 will not have contact with brush contacts 31 or 32. In an alternative variant of this exemplary embodiment, a further contact with the circuit 100 in insulator 28 may be made to establish further electrical efficiencies of the type required for the circuit (not shown) that may be powered by the induced current formed as a result of the embodiments of the present invention.

An exemplary circuit 100 within insulator 28 may be formed of a sink 101 to incoming charge electrically connecting a one-way charge valve 102 (e.g., a diode) in series with a charge collector 103 (e.g., a capacitor), which is in series with an oppositely charged drain switch 104. Thus, a current I flowing into wire end 12 will enter brush contact 32 as I₁ and sink 101 as I₂. The charge flowing into insulator 28 will be prevented from returning to wire end 12 by charge valve 102. Thus all charge from I₂ will be collected at charge collector 103 within insulator 28 for the duration of wire end 12 receiving charge from wire 10's induced current. Once wire end 12 is no longer in contact with brush contact 32, wire end 12 may cease to have an induced current, otherwise any additional induced current will be stored within charge collector 103. Once wire end 11 rotates into contact with brush contact 32, the charge stored from the induced current in wire end 12 will be dissipated at drain site 104 (which is opened once wire end 11 is positioned to receive induced current flowing out of wire end 11) to combine with additional induced charge at wire end 11. Thus, even as hemispheres of commutator 25 may not be in contact with brush contacts 31 or 32 to release charge, circuit 100 in insulator 28 may permit perpetual charge collection during rotation of wire 10 regardless of where each hemisphere of commutator is located in its charging rotation about axis A. In a preferred embodiment, the existence of circuit 100 may allow charge collection while looped wire 10 is precluded from rotating in the fluid stream so as not to waste continuously induced charge. The charge collection processes herein described may be applicable to other circuits formed within commutator 25 (e.g., fluid viscosity measuring circuits, insulin or other drug detection/monitoring circuits, drug administration, cholesterol monitoring, etc.).

In a preferred embodiment, circuit 100 may be fabricated according to any known electronic or microelectronic manufacturing processes known to those skilled in the art. Preferably, circuit 100 may be vapor deposited on a half-slab of wire end 11/12 and insulator 28. The charge valve 102 and charge collector 103 are vapor deposited on the insulator 28 while each of sink 101 and drain 104 are vapor deposited to be in conductive contact with either of commutator 25 portions containing wire ends 11 and 12. Once deposited, another half-slab of wire end 11/12 and insulator 28 are placed on the vapor-deposited half-slab of the same materials (but opposite wire end) and are aligned with one another to form commutator 25.

Table 1 is an exemplary graphical embodiment of the charge accumulation and dissemination of commutator 25 according to the illustrative embodiments of FIGS. 7A and 7B. Table 1 represents the counterclockwise rotation of commutator 25 about axis A according to the exemplary embodiment of the present invention in FIGS. 7A and 7B.

According to Table 1, at time 0 an exemplary commutator 25 of FIG. 7A starts with wire end 12 in contact with brush contact 32 and wire end 11 in contact with brush contact 31. At time 0, the charge at wire end 12 is opposite to that of wire end 11 due to induced current flow in wire 10. It should be understood that the charge depicted in Table 1 is relative based on the induced current flow in wire 10 and no particular sign (positive or negative) is meant to be attached to charge values above or below the time axis. It would be understood that charges above and below the x-axis are oppositely charged. As time progresses from time 0 to time 9, the amount or charge exiting from wire end 12 to brush contact 32 decreases due to charge being stored in charge collector 103 via charge circuit sink 101. When wire end 12 is no longer in contact with brush contact 32 (e.g., when commutator 25 rotates its wire end 12 hemisphere out of contact with brush contact 32), circuit 100 may withhold delivering the stored charge into wire end 11 until commutator 25 has rotated wire end 11 (via a switch) to electrically couple with brush contact 32.

The graphical representation of the charge-withhold sequence of the embodiment of the present invention illustrated in FIG. 7A may occur in Table 1 at time 10 to time 13. From time 10 to time 13, neither wire end 12 nor wire end 11 communicate charge. At time 14, wire end 11 connects with brush contact 32 and wire end 12 connects with brush contact 31, as per FIG. 7B. Simultaneously at time 14, circuit 100 begins to dissipate the collected charge collected by wire 12 current flow during times 0 to 9. At that time however, wire end 12 is oppositely charged to what it was charged at times 0 to 9 due to the induced current in wire 10 after 180 degrees of rotation.

With further reference to FIG. 7B, dissipated charge I_{C} enters wire end 11 at charge drain 104 and adds to the current I entering wire end 11 from wire 10. The continuous dissipation of I_{C} adds to the overall charge entering brush contact 32 over times 14 to 22 until the charge collector 103 is depleted of its charge or wire end 11 moves out of contact with brush contact 32. At time 23 to time 26, the commutator 25 has rotated so that wire end 11 and wire end 12 are not in contact with brush contacts 32 and 31. Times 27 to 36 represent wire end 12 reconnecting with brush contact 32 and wire end 11 reconnecting with brush contact 31 (as illustrated in FIG. 7A). During this time period, circuit 100 may once again collect charge from wire end 12 and dissipate that collected charge at a later point in the commutator 25 cycle (e.g., FIG. 7B).

Referring now to the exemplary embodiment of the present invention illustrated in FIGS. 7C-D, a commutator 25 comprising insulator 28 embodying the circuit 100 may comprise an internal conduction liquid chamber 105 comprising chamber insulator portion 106 and chamber conductor portion 107. The dashed conductor portion 107 of conduction liquid chamber 105 may be filled substantially with conduction liquid such as mercury, sodium ion solution, or other ionized solutions/any other liquid conductor known to those skilled in the art, such as, for example, the conduction liquid disclosed in U.S. Patent Application Publication No. 2009/0273083 (U.S. patent application Ser. No. 12/113,024) published Nov. 5, 2009, the disclosures of which are hereby completely incorporated herein by reference in their entirety. Conduction liquid chamber 105 may be configured to possess a certain volume of conduction liquid so that when commutator 25 rotates due to rotation of wire 10 (e.g., counterclockwise about axis A), conduction liquid may remain substantially in conductor portion 107. According to this embodiment, there may be only a certain volume of conduction liquid in chamber 105 to ensure that at no one time a complete circuit 100 may exist between wire ends 11 and 12 through insulator 28.

In another exemplary embodiment of the present invention illustrated by FIG. 7C, conduction liquid chamber 105 and liquid chamber portions 106 and 107 divide circuit 100 into several components whereby each liquid chamber portion acts as a connectivity gate between circuit 100 components. According to an exemplary embodiment of the present invention in FIGS. 7C-D, conduction liquid is found within conduction liquid chamber portion 107 due to gravity acting on the conduction liquid. As per this exemplary embodiment, as commutator 25 rotates, gravity may keep conduction liquid in the southern hemisphere of commutator 25 thereby only connecting a portion of insulator 28 and the entirety of either wire ends 11 or 12 depending on which portion of commutator 25 is in the same hemisphere as the conduction liquid. In another exemplary embodiment of the present invention in FIGS. 7C-D, conduction liquid may be found in any portion of chamber 105 using variable density insulator liquids in conjunction with conduction liquid in any portion 106 or 107 of conduction liquid chamber 105.

As per the aforementioned embodiment, a sodium ion solution conduction liquid may be found in chamber 105 with an electrical insulator such as, for example, oil. During rotation of commutator 25, higher density conduction liquid located in the northern hemisphere at any given time in the rotation may displace lower density chamber liquid (e.g., oil) to achieve uniform location of conduction liquid in chamber 105 throughout commutator 25 rotation cycle. Alternatively, charge conduction liquid may be magnetized and attracted to either one of magnets 8 or 9. According to this embodiment, charge conduction liquid may migrate through chamber 105 to the portion closest the magnet to which it is attracted.

According to one embodiment, charge drain 104 is divided by conduction liquid chamber portion 106 into drain base 104b and drain access 104a. During the time period in which exemplary embodiment of the present invention in FIG. 7C exists (e.g., Table 1, time 0 to 9 and time 27 to 36), charge I_{IN} entering wire end 11 from brush contact 31 remains in wire end 11 and proceeds through wire 10 as I_{A} due to a lack of conduction liquid in chamber portion 106 electrically connecting drain base 104b to drain access 104a. However, conduction liquid in chamber portion 107 establishes a circuit 100 between insulator 28 and wire end 12. Here, a charge I_{B} from wire 10 diverges within wire end 12 into I₁ and I₂ with I₁ being communicated to brush contact 32 and I₂ being communicated to charge sink 101. Charge sink 101 communicates the charge of I₂ from sink base 101b to sink access 101a through the conduction liquid in conduction liquid chamber portion 107. As described above with reference to FIGS. 7A and 7B, circuit 100 components charge valve 102 and charge collector 103 collect a portion of charge I_{B} for dissemination at a later point in the rotation of commutator 25 (e.g., Table 1, time 14 to 22).

In FIG. 7D, commutator 25 has rotated into position where wire end 12 connects with brush contact 31 and wire end 11 connects with brush contact 32. In this arrangement, commutator 25 may use any number of the conduction liquid moving techniques (e.g., gravity, variable density displacement) to place conduction liquid in conduction liquid portion 107. Once located there, charge I_{B} entering wire end 11 from wire 10 will receive charge I_{C} disseminated by circuit 100's charge collector 103 according to the other aforementioned embodiments of the present invention in FIG. 7B and Table 1, time 14-22. Charge I_{C} may be permitted to enter wire end 11 in commutator 5 only because conduction liquid may be substantially located between charge drain access 104a and charge drain base 104b. A complete discharge circuit thereby forms due to presence of the conduction liquid in conduction liquid chamber portion 107. The resulting outgoing charge I received at brush contact 32 is a combination of collected charge I_{C} and I_{B} (induced from wire 10). This resulting outgoing charge may be graphically realized in Table 1, time 14-22 for wire end 11. As no conduction liquid may be located in conduction liquid chamber portion 106, no further charge accumulation may be had through charge drain 104 in wire end 12 (as represented in Table 1, time 14-22 for wire end 12). Thus, charge I_{IN} entering wire end 12 will continue through commutator 25 as charge I_{A} entering wire 10. In one embodiment of the present invention according to FIG. 7D, charge I_{IN} and I_{A} may be substantially the same.

It may be appreciated that circuit 100 may be the circuit to which wire ends 11 and 12 electrically couple and to which induced current in wire 10 flows. In such an arrangement, circuit 100 may also comprise resistors, capacitors, transistors, diodes, amplifiers, inductors, transformers and other known electrical components. According to this embodiment, a functioning circuit may exist without need for brush contacts, slip rings, or wires to an embedded circuit elsewhere in prosthesis 4, e.g., the circuit is embedded on wire 10. A circuit 100 constructed according to the embodiments herein described may be self-contained and powered within a commutator, within wire 10, etc., without resort to external electrical components but for wire 10 and a magnetic field.

An exemplary wire 10 circuit 100 may be illustrated in FIG. 7E. Circuit 100 may comprise current points 109/110, current bridge 111/112 and elements 115. Current entering from wire 10 into wire end 12 of commutator 25 may enter current point 109 and pass through current bridge 112. Circuit elements 115 may be any known electrical components to form circuits. In the illustrative embodiment of the present invention in FIG. 7E, circuit elements 115 have been arranged to form an RLC-circuit which is grounded within insulator 28. Due to insulator 28 being unable to conduct electricity it is a preferred grounding cite for circuit 100 and its elements 115. However, it may be preferable to ground circuit 100 to a portion of body 7 in which it is lodged in either cavity 75 or bottleneck space 74.

Circuit 100 may have current flowing into it in substantially one-direction by virtue of the current bridges 111 and 112 (e.g., diodes or other current direction restrictors known to those skilled in the art). Alternatively, bridges 111 and 112 may alternate which current from which wire end (11 or 12) may enter circuit elements 115. For example, as a looped wire 10 rotates counterclockwise about axis A in a magnetic field perpendicular to the axis A and in a direction opposite that of the rotation, wire end 12 may receive induced current flowing toward current point 109. As induced current from wire 10 enters current point 109 it is transmitted to at least one circuit element 115 through bridge 112 connecting the conductive portion 12 of commutator 25 to the at least one circuit element 115 disposed within the insulator portion 28 of commutator 25. Upon exiting the at least one circuit element 115 the induced current traverses bridge 111 and is not permitted reentry upon exiting current point 110. This may be so because bridge 111 only permits current to exit from circuit element 115 and not return by flowing through current point 110. Similarly, conductive fluid chamber embodiments disclosed with respect to FIGS. 7C and 7D may be equally applicable to the wire 10 circuit 100 of the embodiments related to FIG. 7E to effect similar current control. Alternatively, bridges 112 and 111 may themselves be timed to allow alternating current flow directions by using other circuit elements (e.g., bias switches) or the magnetic field. Those skilled in the art would understand methods and mechanisms to implement switching electrical devices to allow different directional currents to traverse any given point in a circuit in a given time and that the same may be applied in practice to the current bridges 111 and 112 of the present invention.

Moving now to the illustrative embodiment of the present invention in FIG. 8, commutator 25 may be rotatably coupled to platform 7 and electrically coupled to brush contacts 31 and 32 within platform 7. As previously mentioned, commutator 25 may be rotatably coupled within platform 7 through numerous methods similar to those employed to rotatably couple wire ends 11 and 12 to slip rings 21 and 22, respectively or collectively. As a uniform structure of wire end 11, insulator 28 and wire end 12, commutator 25 lends itself to other forms of rotatable and electrical coupling.

In one embodiment, commutator 25 may be electrically coupled to brush contacts 31 and 32 within a bottleneck cavity 74 in platform 7. Neck 76 of bottleneck cavity 74 may preclude inoperative displacement of commutator 25 within platform 7 and may retain commutator 25 in contact with brush contacts 31 and 32. In forming bottleneck cavity 74, platform 7 may be fabricated from a plastic or fabric material molded about a metal bit or frame. Alternatively, platform 7 may be made out of a stretchable biocompatible material so as to allow tight formation of bottleneck cavity 74 but permit expansion of neck 76 to allow commutator 25 to enter. Alternatively, platform 7 may be made of a sewable or heat bondable biocompatible material that may contain brush contacts 31 and 32. The sewable material may be folded, wrapped, stretched, etc. to encapsulate the commuter 25 so that brush contacts 31 and 32 contact the commutator 25 surface during rotation. Once encapsulated, the biocompatible material may be sewed in a plane containing rotation axis A to form bottleneck cavity 74 exposing only wires ends 11 and 12 leading out to rotating portions of looped wire 10. Where a material may not be sewable, it may be joined to other material through heat bonding, adhesive or other material adhesion means known to those skilled in the art. In the aforementioned arrangement, the circuit wires 41 and 42 may be embedded in the platform 7 fabric prior to encapsulation or be connected to brush contacts 31 and 32 post-encapsulation. Bottleneck cavity 74 may be any form of containment space for commutator 25 that comprises at least one rotation permissible surface in which the commutator 25 is held (e.g., a brush contacts ring, a molded form, etc.) This configuration is in contrast to a socket 75 which may comprise any type of surface.

According to another embodiment, commutator 25 of FIG. 8 may possess the circuit 100 and its respective operation described in FIGS. 7A-D. In another embodiment, brush contacts 31 and 32 may be embedded within a portion of platform 7. This portion of platform 7 may blanket commutator 25 in portions contacting a plane containing rotation axis A permitting rotatable and electrical coupling of commutator 25 with embedded contacts 31 and 32. Neck 76 may be formed by sewing those parts of the platform 7 blanket enclosing commutator 25 to each other leaving an opening for looped wire 10 ends 11 and 12. According to this embodiment, the sewed portion of platform 7 leaves open a neck 76 that precludes commutator 25 from exiting bottleneck cavity 74.

Referring to the exemplary embodiment of the present invention in FIG. 9, spherical commutator 25 rotates within a socket 75 in platform 7. Additionally, cuff 77 may prevent spherical commutator 25 from displacing during operation of the various embodiments of the present invention. Socket 75 and cuff 77 may be made of any flexible and expandable biocompatible materials that allow for close fits about spherical commutator 25 yet permit its insertion post formation. Socket 75 may also be made from fabric material according to the blanket cavity 74 embodiment described with respect to other embodiments of the present invention. Accordingly, socket 75 may comprise a blanket of platform 7 material with embedded brush contacts 31 and 32. The blanket of platform 7 encapsulates spherical commutator 25 to allow rotatable and electrical contact therewith. Sewing, heat bonding or adhesively sealing the ends of blanket platform 7 to one another within a plane containing rotation axis A, the space allowing for looped wire 10 ends 11 and 12 to pass may be serve as the cuff 77 of the blanket platform 7 socket 75 according to this exemplary embodiment.

In an alternate embodiment, covering 27 of looped wire 10 may also form a wall that fits within either cuff 77 or neck 76 of the embodiments of the present invention according to FIGS. 8 and 9. Covering 27 wall may shield commutator 25 from oncoming fluid that may try to enter cavity 74 or socket 75. Alternately, covering 27 wall may prevent displacement of commutator 25 in cavity 74 or socket 75 or may preclude commutator 25 from exiting cavity 74 or socket 75. In another alternative embodiment, covering 27 wall may be structured within a groove within a cuff 77 or neck 76 that creates a substantially hermetic seal in which wire ends 11 and 12 operate. Alternatively, the seal may just prevent leakage of tissue fluid into cavity 74 or socket 75, but not be completely airtight.

In a preferred embodiment, commutator 25 may rotate due to rotational forces on wire 10 within socket 75 located in any part of prosthetic 4. Exposed portions of brush contacts 31 and 32 touch the respective wire end surfaces 11 and 12, respectively, of commutator 25 from within socket 75 during commutator 25 rotation. Socket 75 may possess natural inoperative displacement control of commutator 25. Socket 75 may also be useful in withstanding turbulent fluid flow rotations of wire 10 due to its accommodating shape for spherical commutator 25. According to one embodiment, spherical commutator 25 of FIG. 9 may possess the circuit 100 and its respective operation described in the embodiments herein. In an alternative embodiment, spherical commutator 25 may possess numerous circuits 100 according to the embodiments described herein, e.g., those depicted in FIGS. 7A-D.

It might also be appreciated by those skilled in the art that a commutator 25 according to the exemplary embodiments of FIGS. 4, 5, 7A-D, 8 and 9 may be used partially within a prosthetic 4 coupled to tissue 3. Alternatively, a commutator 25 of the embodiments described herein may not be limited to any particular use, and may be utilized in other applications (e.g., micro-electronic applications, commercial electronic applications, automobiles, aviation equipment, motors, generators, etc.)

The illustrative embodiment illustrated in FIG. 10 depicts a cross-sectional view of platform 7 holding rotor 5 on an axis of rotation A. Rotor 5 freely rotates in incoming stream 52 within aperture 16. The charge section 2 is not shown but may be understood to connect wire 10 embedded within rotor 5 to circuit wires 41 and 42. It should be understood that although FIG. 10 shows rotor 5 receiving stream 52, that wire 10 may also receive stream 52 without having to be embedded within rotor 5. Therefore, according to the embodiments of FIG. 10, rotor 5 may be interpreted as either rotor 5 with wire 10 embedded there within or wire 10 without rotor 5. Similarly, the curved shape of rotor 5 depicted in FIG. 10 may likewise be possessed by wire 10 without being embedded in rotor 5 of the same curvature.

With reference to FIG. 10, prosthetic 4 may be medically attached to tissue 3. Tissue 3 may abut prosthetic 4 or enclose prosthetic 4 so that any fluid 50 flowing in tissue 3 may flow within prosthetic 4. The portion of fluid 50 flowing through platform 7 is fluid stream 52. Stream 52 impacts rotor 5 on the surface facing oncoming stream 52. The rotation imparted by contact of stream 52 with rotor 5 rotates either rotor 5 with looped wire 10 embedded there within or exclusively looped wire 10 depending upon which of the disclosed embodiments of the present invention receive stream 52. In one exemplary embodiment of the present invention illustrated in FIG. 10, looped wire 10 may be looped within rotor 5 and curved into a substantial “S” shape. Other loop and bend configurations for wire 10 disclosed in other embodiments (e.g., FIGS. 12A-B, 13A-L, 14A-B and 15A-B) may also be contemplated for wire 10 in the illustrative embodiment of the present invention in FIG. 10. Similarly, rotor 5 may cover any and all contemplated looped wire 10 configurations previously discussed. Structures of rotor 5 discussed in other embodiments disclosed herein may also be considered applicable to the embodiment illustrated by FIG. 10.

In the cross-sectional view of several embodiments of the present invention depicted in FIG. 10, magnets 8 and 9 are located opposite one another with aperture 16 disposed between them. According to the embodiments of the present invention represented in FIG. 10, one of either magnet 8 or 9 may be slightly farther from tissue interface 6 than the other magnet. Additionally, axis A may be substantially parallel with tissue interface 6. According to another embodiment of the present invention disclosed in FIG. 10, aperture 16 may be a carved out portion of platform 7 allowing fluid 50 and streams 52 to pass there through. Platform 7 may be positioned to achieve optimal use of stream 52 flow on rotor 5 and looped wire 10. According to one embodiment of the present invention, platform 7 may be curved or cupped to catch as much incoming streams 52 in fluid flow 50 within tissue 3. Platform 7 may be shaped to induce stream 52 to target a particular section of rotor 5 (e.g., having ramps, channels or conduits for all or a portion of stream 52 on its stream-facing surface). Similarly, aperture 16 may also be shaped to induce a higher velocity stream 52 impacting upon rotor 5 and/or looped wire 10 (e.g., throttle cavities, manifold shapes, etc.). Those skilled in the art would understand that both platform 7 and aperture 16 may be shaped in any way that produces optimal fluid stream 52 to impact on rotor 5 and/or looped wire 10 in accordance with the embodiments of the present invention. Platform 7 may extend toward and may additionally contact and/or integrate with other portions of prosthesis 4 maintaining biologically suitable clearance for passage of fluid 50.

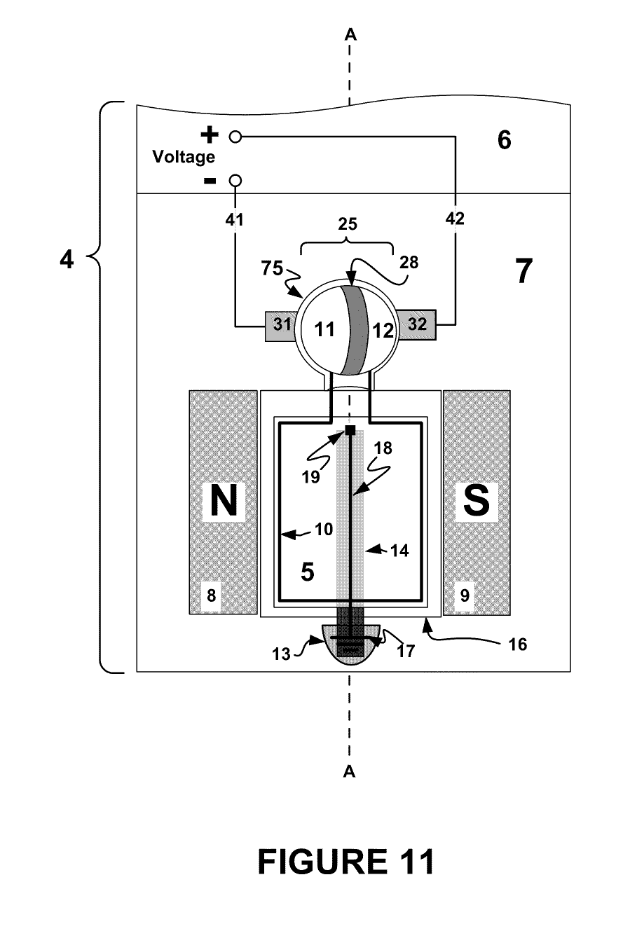

FIG. 11 depicts an exemplary embodiment of the present invention where the axis of rotation A may be either substantially perpendicular to tissue interface 6 or may be rotated 90 degrees from its position in other embodiments of the present invention. Though depicted as rotor 5 containing an embedded looped wire 10, it may also be understood, without departing from the spirit and scope of the present invention, that the embodiments of FIG. 11 may also relate to looped wire 10 without rotor 5 comprising it. Like the embodiments of the present invention in FIG. 2, prosthetic 4 may contain tissue interface 6 in substantial contact with tissue 3 (not shown). The portion of prosthetic 4 with highest amount of contact with tissue fluid 50 may be platform 7. Platform 7 may hold rotor 5 and/or looped wire 10 according to any of the structural supports described with relation to the embodiments disclosed herein.

In an exemplary embodiment according to FIG. 11, holder 14 (in conjunction with wire ends 11 and 12) may couple wire 10 to platform 7. According to this embodiment of the present invention, holder 14 may substantially enclose support 18 and anchor 19. Additionally, holder 14 may substantially embody ground 17. Pocket 13 may be configured to receive holder 14 within platform 7. Holder 14 may be manufactured from any biocompatible material known to those skilled in the art and may be molded into an exemplary rotor 5 or attached to looped wire 10 or looped wire 10 covering 27. According to this exemplary embodiment, usage of holder 14 may enhance rotation stability of wire 10 and/or rotor 5 in the face of turbulent streams 52 within fluid flow 50 in tissue 3. Holder 14 may be utilized with any other disclosed embodiments and variations thereof according to the description of the operations of the present invention.

According to FIG. 11, commutator 25 may be shaped in any way according to the various embodiments of the present invention. An exemplary commutator 25 according to the present invention illustrated in FIG. 11 may be spherical in shape and embedded in platform 7 within a bottlenecked socket 75. In this way, spherical commutator 25 may rotate freely in response to oncoming fluid streams 52 without substantial displacement of commutator 25 from brush contacts 31 and 32. Insulator region 28 may be formed according to the previous embodiments of the present invention, but an exemplary insulator region 28 may be a lightweight ceramic, composite or plastic. Brush contacts 31 and 32 may be shaped according to the shape of bottlenecked socket 75 to ensure internal displacement of commutator 25 within socket 75 due to oncoming streams 52. According to one embodiment, streams 52 may cause distal displacement of looped wire 10 and proximal displacement of commutator 25 within socket 75. Brush contacts 31 and 32 may be shaped accordingly to continue to collect charge from commutator 25 despite formation of space in socket 75 by virtue of commutator 25 being displaced (e.g., contacts 31 and 32 shaped as arcs that are positioned about the inside of socket 75 to maintain contact during any displacements).

Turning now to the exemplary embodiment of the present invention according to FIG. 12A, wire 10 may be both looped and bent. This illustrative embodiment of the present invention may be utilized within any of the other embodiments described herein. Accordingly, a bent and looped wire 10 may be found within rotor 5 or be substantially covered with covering 27.

According to one exemplary embodiment depicted in FIG. 12A, looped wire 10 may be bent in a substantial “S”, “H” or “X” shape and may be covered across all or a portion of its loopings with cover 27. Other looped wire configurations useable in this version of the exemplary embodiment of FIG. 12A may be observed in FIGS. 13A-L. The amount of bending 62 is measured in degrees from the axis of rotation A. The bending 62 may be used to characterize the deviation of a portion of looped wire from the axis of rotation A. In FIG. 12B, intermediate portions of looped wire 10, α, β, γ and λ may experience different degrees of bends 64, 65, 66 and 67, respectively. Wire 10 portions α and β may be considered “base” portions of looped wire 10 that are in closest proximity to the axis of rotation A. Wire 10 portions γ and λ are the “branch” portions of looped wire 10 that stem from base portions α and β, respectively. According to the exemplary embodiment of the present invention according to FIG. 12B, bending of branches γ and λ may be characterized as the number of degrees from axis A plus displacement from the axis line or degrees from the respective base portion of looped wire 10. It may be understood that other looped wire 10 configurations may have any number of base portions and branches. Although the exemplary embodiment of the present invention in FIG. 12B may be bent by degrees in one plane, it should be understood that the bends of wire 10 may occur in any plane oriented in three-dimensional space (e.g., FIGS. 13C, 13E, 13G-H and 13J-K).

In a preferred embodiment, a looped wire 10 may have a configuration having length of segments γ or λ=2×(length of segments α or β) and angles 66/67=90°+½×(angles 65/64) for turbulent fluid flows in tissue 3 and length of segments γ or λ=length of segments α or β and angles 66/67=64/65 for laminar flows. The dimensioning of segments γ, λ, α and β may be developed to avoid eddy currents of tissue fluid occurring in the aperture 16.

FIGS. 13A-L depict a selection of loop configurations for looped wire 10 having wire ends within the same locus L of operation (e.g., the wire ends are adjacent one another in three-dimensional space to connect looped wire 10 to a charge section 2 according to the exemplary embodiments of the present invention). FIG. 13A depicts an exemplary discrete wave looped wire 10 with undulations at right angles. FIG. 13B depicts an exemplary substantially sinusoidal wave looped wire 10. FIG. 13C illustrates an exemplary discrete wave looped wire with a single underpass leading a wire end to the operative locus L. It should be noticed that the underpass portion of wire in FIG. 13C requires bending the looped wire 10 in alternative planes in three-dimensional space to achieve positioning of wire ends 11 and 12 at a locus L in the same or proximal plane. FIG. 13D is an exemplary elongated looped wire 10 whose wire is longest at those portions that substantially cut the magnetic field between magnets 8 and 9. FIG. 13E illustrates an exemplary looped wire body that encloses a geometric space perpendicular to the axis of rotation A. According to one embodiment of the present invention illustrated in FIG. 13E, the looped wire body maybe a cube or box. In another embodiment, the looped wire body may be an ellipsoid or a sphere (see, e.g., FIG. 13J). FIG. 13F is an exemplary rounded loop configuration of looped wire 10.

FIGS. 13G-H and 13J-K illustrate an exemplary multi-planar wire looping for receiving charge throughout the rotation of the looped wire 10 about axis A. According to these exemplary embodiments of the present invention, charge may continuously be received within a rotating wire 10. More particularly, charge may be received at odd time periods (13G) or even time periods (13H, J and K) depending on which planes a portion of or looping of wire resides. In one exemplary embodiment of the present invention according to FIGS. 13A-L, charge may be induced in those portions of looped wire 10 that are perpendicular to the magnet field between magnets 8 and 9. In another exemplary embodiment, charge is induced in those portions of looped wire 10 looped in the same or a parallel plane.

FIGS. 13I and 13L illustrate exemplary labyrinthine wire looping according to the present invention. Labyrinthine looped wire 10 may permit increased charge creation in wire 10 in single or multiple plane configurations. In an alternative embodiment, the spacing of adjacent segments of wire in the labyrinthine loops permit additional dragging by the fluid flowing through the labyrinthine loops in covered or uncovered form. According to this alternative embodiment, the spacing of the labyrinthine loops take advantage of the cohesive forces of fluids within tissue 3. The labyrinthine configurations depicted in FIGS. 13I and 13L may be incorporated into other embodiments of the present invention illustrated in FIGS. 13A-H and 13J-K.

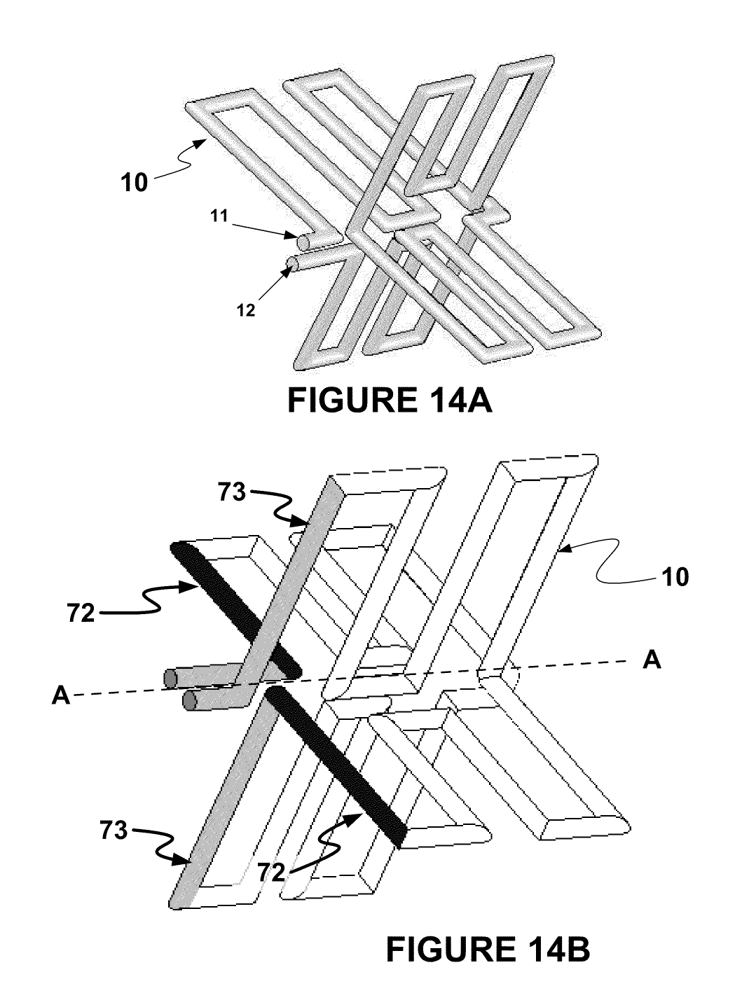

FIGS. 14A and 14B illustrate another embodiment of the present invention in which looped wire 10 occupies multiple planes. FIG. 14A illustrates looped wire 10 with wire ends 11 and 12 with operations, capability and functionality as described with respect to the other embodiments of the present invention. FIG. 14B is an illustrative embodiment of multi-planar charge induction according to the operations of the embodiments of the present invention. Rotation axis A acts as the intersection for two perpendicular planes in multi-planar looped wire 10. Loop sections 72 occupy one plane that is perpendicularly intersected by loop sections 73. In one embodiment, loop sections 72 receive induced current from the magnetic field of magnets 8 and 9 while loop sections 73 do not receive induced current. In another embodiment, magnets 8 and 9 are spaced and formed such that different induced currents exist in looped sections 72 and 73 at different time intervals. According to the embodiments of the present invention in FIGS. 14A and 14B, multi-planar wire loop 10 may have looped wire occupying multiple planes all connected with the same wire ends, e.g., wire ends 11 and 12. Existence of wire in multiple planes may increase the propensity for induced current to exist in the total looped wire at any given point in time. As will be described below, constant reception of reduced current may be handled by particular placement of brush contacts about the electrical coupling points of a multi-planar wire so as to receive consistent charges in AC current systems and DC current systems described herein.

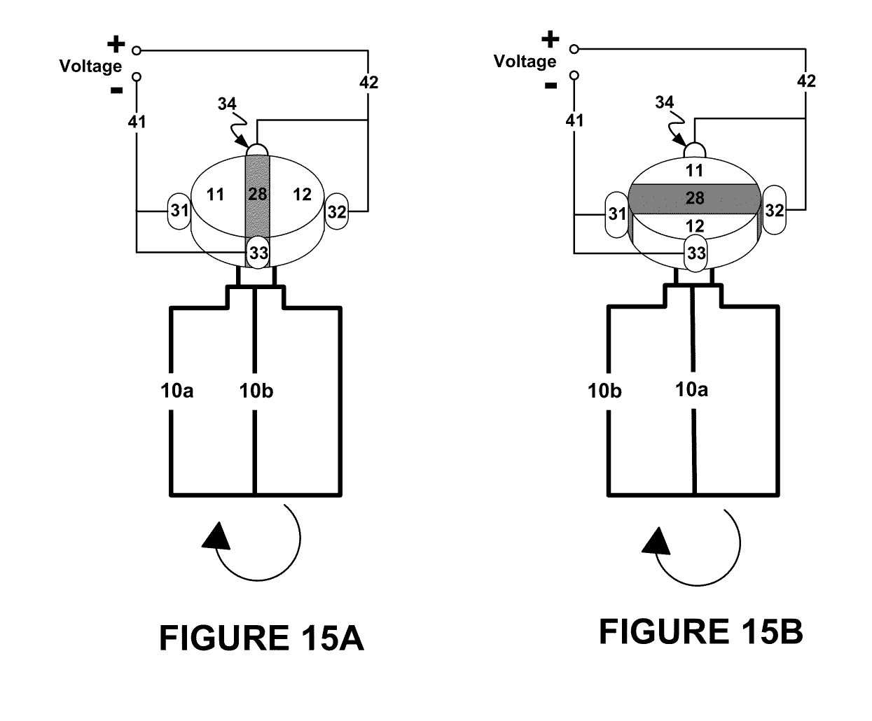

FIGS. 15A and 15B illustrate an embodiment of the present invention in which multi-planar looped wire 10 may continuously contribute charge to circuit wires 41 and 42. FIG. 15A illustrates multiple planed looped wire 10. According to the illustrative embodiment of FIG. 15A, looped wire 10 may be dual-planar with loop planes 10a and 10b. Looped wire 10 may have any number of loop planes according to the embodiments of the present invention. In FIG. 15A, brush contacts 31-34 are spaced according to the number and angle of planes in looped wire 10. In one embodiment, brush contacts 31-34 are spaced 90 degrees apart while wire loop 10 planes are spaced 90 degrees apart. In another embodiment according to FIG. 15A, brush contacts 31-34 may be spaced 60 degrees apart while wire loop 10 planes are spaced 60 degrees apart. Other space configurations may be appreciated to encourage constant reception of charge at any one of brush contacts 31-34 during rotation of looped wire 10.

According to the embodiment of the present invention illustrated in FIGS. 15A and 15B, wire ends 11 and 12 may remain connected to their respective portions of commutator 25. At the point in time depicted in FIG. 15A, looped wire plane 10a receives an induced current and looped wire plane 10b does not receive an induced current. At that time, charge flows through wire ends 11 and 12 to be received by brush contacts 31 and 32. At the point in time depicted in FIG. 15B, looped wire plane 10b receives an induced current and looped wire plane 10a does not receive an induced current. At that time, charge flows through wire ends 11 and 12 to be received by brush contacts 33 and 34. In both of the embodiments illustrated in FIGS. 15A and 15B, the spacing of brush contacts 31-34 and corresponding inclination of looped wire planes 10a and 10b maintain constant flow of induced current to the circuit wires 41 and 42. According to the embodiments illustrated in FIGS. 15A and 15B, coordinated angular spacing of brush contacts with that of the looped wire planes permits a continuous and unhindered reception of induced current in charge section 2.

Tables 2 and 3 may illustrate the variation in charge reception due to alignment and misalignment of contacts with the planar loops of multi-planar looped wire 10.

Table 2 may illustrate the charge reception over a revolution of multi-planar looped wire 10 for wire loops 10a and 10b oriented in planes at 90-degrees from one another with each pair of their respective contacts 31/32 and 33/34 oriented at 0-degrees and 180-degrees and 90-degrees and 270-degrees respectively.

Table 3 may illustrate the charge reception over a revolution of multi-planar looped wire 10 for wire loop planes 10a and 10b oriented at 90-degrees from one another with contacts 31/32 oriented at 0-degrees and 180-degrees and contacts 33/34 oriented at 75-degrees and 255-degrees respectively.

It may be appreciated that a positioning arrangement of contacts and wire loop planes of the type illustrated in Table 3 may enable stronger AC current generation. Alternatively, as the number of looped wire planes increase to occupy the vacant angular locations between looped wires 10a and 10b (e.g., additional planar loops of wire at 45-degrees and 135-degrees) there may be a stronger DC current generation according to the embodiment depicted by Table 3.

In a preferred embodiment, coordinated spacing of brush contacts and planar angling of looped wire achieves optimal DC charge flow. Coordinating the angular location of a number of brush contacts about a commutator whose wire ends connect to a looped wire with an equal number of loops in equally angularly oriented number of planes allows for constant, consistent DC current reception into the circuits of the present invention.

In another preferred embodiment, uncoordinated spacing of brush contacts and planar angling of multi-planar looped wire may create AC charge flow in an otherwise DC charge induced system (e.g., by off-setting the angular orientation of the brush contacts from the orientation of multi-planar looped wire). It may also supplement the AC current system already in place in the circuit of the present invention, e.g., in embodiments possessing slip rings instead of a commutator.

In a preferred embodiment, prosthesis 4 may be a stent-graft of which platform 7 is an interior portion. Looped wire 10 may be made of Nitinol metal so as to be compressed within a catheter used to deploy stent-graft 4 within an artery or vein 3. Looped wire 10 is a unitary plane labyrinthine configuration whose ends are part of a commutator 25 nested within a bottlenecked socket 75 in platform 7. Separating either of the labyrinthine wire ends is a PVC plastic of a thickness equal to one quarter the thickness of the average thickness of wire ends 11 and 12. Brush contacts 31 and 32 and circuit wires 41 and 42 are embedded within platform 7 and connect to a circuit device also embedded in stent-graft 4. Bendable sheet magnets 8 and 9 are also embedded within platform 7. According to this preferred embodiment, prosthesis 4 and all wires and magnets connected to or embedded therein can be compressed on a guide wire and deployed within an artery or vein 3. Once deployed, blood passes through and around labyrinthine looped wire 10 and causes rotation by fluid flows against and through the segments of looped wire. Such blood flow pushing looped wire 10 induces current in the segments of looped wire 10 that cut through the magnetic field of magnets 8 and 9. In this preferred embodiment, a charge section 2 receiving blood flow over and through looped wire 10 will continuously charge an attached circuit as long as there is blood flow in the organism in which it is deployed.

Circuit wires 41 and 42 may charge circuits of any form or capability. One type of circuit that may be utilized may be an implantable sensor, such as for example, those of the type found in U.S. Pat. Nos. 7,232,460, 7,452,334 and United States Patent Application Publication No. 2002/0128546 (U.S. patent application Ser. No. 10/041,036) published Sep. 12, 2002, the disclosures of which are completely incorporated herein by reference in their entirety. In one embodiment, the circuit connected to wires 41 and 42 measure contents of the fluid passing through tissue 3. In one preferred embodiment, a blood constituency measuring apparatus of the type known to those skilled in the art may be charged by charge section 2 and operated by the continuous charging, e.g., an insulin measuring and deployment system that alerts an individual of low insulin levels internally. In another preferred embodiment, an electrically timed releasing circuit for release of a drug may also be utilized with the charge section 2 disclosed in the other embodiments of the present invention (e.g., signal receiving drug dispensation apparatus).

In another preferred embodiment, charge section 2 may utilize a portion of its charge to stimulate constant expansion of a stent to which the charge section 2 is connected. Circuit wires 41 and 42 are not limited to circuits within the deployment cite of the prosthesis but may charge other devices external to the deployment cite, e.g., wires 41 and 42 going from charge section 2 in prosthesis 4 within aorta to surface of atrium where pace-maker or artificial heart is located. Those skilled in the art will recognize the present invention may act as the charge site and/or power source for any form of circuit with the purpose of treatment, monitoring, control and affecting the life system of the living organism in which it is implanted.

According to another preferred embodiment, blood flow through looped wire 10 within a luminar tissue 3 may be disorganized and filtered as it passes through and in between the loops of looped wire 10. One advantage to such a system may be the decrease in plaque build-up within a vessel by scattering its presence in the fluid flowing through the looped wire 10. Another advantage to such a system may be to mix drug solute within the fluid stream. In this preferred embodiment, the existence of charge section 2 or electrical coupling is not material to the aforementioned advantages, but may nevertheless by added to the system to obtain additional advantages.

According to another preferred embodiment, wire 10 may be covered with a drug D or other form of biologically suitable substance that may react when a charge is induced in wire 10. Accordingly, the combined induction of current and coating of drug D may create a system whereby the combined capabilities of the disclosed invention can both activate and disseminate drug into the tissue of an organism. Non-dynamic signal-activated drug deployment may be shown, for example, for drugs used in the systems in U.S. Patent Application Publication No. 2010/0010148 (U.S. patent application Ser. No. 12/442,959) published Jan. 14, 2010, the disclosures of which are completely incorporated herein by reference in their entirety. Similarly, signal and electrical pulse induced drug delivery may also be accomplished by way of the present invention operation according to the disclosed embodiments herein.

The circuits and electrical signal apparati of the disclosed embodiments may be similarly located exclusively within commutator 25 of the present invention. According to this embodiment, a circuit 100 may possess any combination of electrical components 115 receiving induced current directly from wire 10 (e.g., at points 109 or 110 depending on the permitted current flow). In this way, the electrical systems powered by the operation of electrical/mechanical process of the present invention could be self-enclosed in a commutator without additional wiring in the prosthesis 4. For example, the sensor circuits described herein could be located within the insulator 28 with charge entry and exit portions in wire ends 11 and 12 of commutator 25. Other circuits useful in the maintenance, monitoring and analysis of the internal operations of the organism may also be found within the commutator 25 according to the embodiments of the present invention including, but not limited to, FIGS. 7A-E.

Many further variations and modifications will suggest themselves to those skilled in the art upon making reference to the above disclosure and foregoing illustrative embodiments, which are given by way of example only, and are not intended to limit the scope and spirit of the interrelated embodiments of the invention described herein.

1. A device, comprising:
a body implanted in an organism; and,
a looped wire rotatably coupled to said body, said looped wire rotating due to fluid flow through said organism.

2. The device of claim 1 wherein said wire comprises a drug.

3. The device of claim 2 further comprising a means for releasing said drug.

4. The device of claim 1 wherein said wire is electrically conductive.

5. The device of claim 4 further comprising oppositely polarized magnets separated by at least a portion of wire.

6. The device of claim 5 further comprising a circuit comprising at least one circuit element coupled to said wire.

7. The device of claim 5 wherein said body comprises at least one conductor rotatably coupling said looped wire to said body.

8. The device of claim 5 wherein rotation of said looped wire sends current into said body.

9. The device of claim 5 wherein rotation of said looped wire sends current through a circuit made up of said wire.

10. The device of claim 9 wherein said wire further comprises a releasable drug released by at least said current.

11. The device of claim 6 wherein said current enters at least one circuit element in said body.

12. The device of claim 6 wherein said current enters at least one circuit element in said wire.

13. The device of claim 7 wherein said at least one conductor is a plurality of rings at least one of which is a conductor.

14. The device of claim 7 wherein said at least one conductor is a plurality of rings at least two of which are a conductor.

15. The device of claim 12 wherein said at least one circuit element responds to conditions of said fluid.

16. A device, comprising:
a cavity made in a prosthesis; and,
a looped wire rotatably coupled within said cavity and rotating due to fluid flowing against said prosthesis.

17. The device of claim 16 wherein said looped wire comprises a releasable drug.

18. The device of claim 16 wherein said prosthesis comprises means for inducing charge in said looped wire.

19. The device of claim 18 further comprising a circuit electrically coupled to a portion of said looped wire within said cavity.

20. The device of claim 16 wherein said looped wire comprises a releasable drug, said prosthesis comprises means for inducing charge in said looped wire, said induced charge assisting release of said drug.
